SEQUENCE LISTING (4 sequences)

SEQ ID NO: 1 - DNA, 15 bp, Artificial Sequence
  Description of Artificial Sequence Synthetic oligonucleotide NH2~OD1
acgtcaggtggcact

SEQ ID NO: 2 - DNA, 20 bp, Artificial Sequence
  Description of Artificial Sequence Synthetic oligonucleotide OD2
agtgccacctgacgtctaag

SEQ ID NO: 3 - DNA, 20 bp, Artificial Sequence
  Description of Artificial Sequence Synthetic oligonucleotide Diphenyl-OD2
agtgccacctgacgtctaag

SEQ ID NO: 4 - DNA, 20 bp, Artificial Sequence
  Description of Artificial Sequence Synthetic oligonucleotide OD3
cgtacatgctagtcagtcag

(19) United States
(12) Patent Application Publication
(10) Pub. No.: US 2004/0072212 A1
(43) Pub. Date: Apr. 15, 2004

(54) Recognition-driven alkylation of biopolymers

(76) Inventors: Steven E. Rokita, Silver Spring, MD (US); Qibing Zhou, Glen Allen, VA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/455,805

(22) Filed: Jun. 6, 2003

Related U.S. Application Data
(60) Provisional application No. 60/387,061, filed on Jun. 7, 2002.

(51) Int. Cl. C12Q 1/68

(52) U.S. Cl. 435/6

(57) ABSTRACT

The present invention relates to selective, target-activated modification of target molecules. The present invention also relates to methods and reagents for the selective, target-activated modification of target molecules. The present invention further relates to intermediates in the selective, target-activated modification of target molecules.

This application claims the benefit of U.S. Provisional Application Serial No. 60/387,061, filed Jun. 7, 2002, the disclosure of which is hereby incorporated by reference herein in its entirety.

### FIELD OF THE INVENTION

This invention relates to methods for selective, target-activated modification of target molecules. The invention further relates to reagents and intermediates in the target-activated alkylation of target molecules.

### BACKGROUND OF THE INVENTION

The selective modification of biological targets (e.g., DNA) has been of longstanding interest due in part to the breadth and diversity of its application to medical research. However, currently the lack of target specificity of agents acting at the level of DNA is problematic. For example, while the intrinsic chemical reactivity of many agents may be used to distinguish between general types of amino acid chains or nucleobases, rarely can such agents identify individual sites of interest. This lack of target specificity severely limits the therapeutic potential of such agents.

Nevertheless, the modification of biological targets continues to hold great potential as a tool for molecular scientists. The modification of biological targets with alkylating agents involves reactive electrophiles that are either intrinsic, generated in situ or activated by the target. Intrinsic electrophiles react spontaneously with nucleophiles and the nucleophiles are alkylated based upon nucleophilic strength. While intrinsic alkylation provides no target specificity under biological conditions, this has been improved by employing target-binding affinitive functionalities. However, a major disadvantage of intrinsic electrophiles is the competitive quenching of electrophiles by cellular proteins and DNA as nucleophiles compete for target recognition.

Electrophiles generated in situ are derived from compounds which are stable under physiological conditions until activation through metabolic pathways or initiation by signals. Such in situ generation effectively reduces the potential degradation of reactive electrophiles before target recognition, and thus affords a high efficiency of target alkylation. However, quenching of the resulting electrophiles by the cellular nucleophiles persists as major competition.

In contrast, target-activated electrophiles are the ideal alkylating agents since they react selectively with the target. This target-specific modification is accomplished by activating latent reactive electrophiles through target recognition. One area that provides examples of such electrophiles is the modification of DNA by natural products and their derivatives. Gates K. S. In; Chaires, J. B., Curr. Opin. in Struct. Biol 1998, 8, 314-320; Rajski, S. R, Chem. Rev. 1998, 98, 2723-2795; Gniazdowski et al., Chem. Rev. 1996, 96, 619-634. For example, it has been demonstrated that intrinsic nitrogen mustard derivatives spontaneously alkylate adenines and guanines of duplex DNA under physiological conditions. Osborne et al, Chem. Res. Toxicol. 1995, 8, 3 16-320; Rink et al., J. Am. Chem. Soc. 1993, 115, 255 1-2557. A nitrogen mustard oligonucleotide conjugate has been shown to alkylate both strands of duplex DNA sequence-specifically from the major groove. Kutyavin et al., J. Am. Chem. Soc. 1993, 115, 9303-9304; Reed et al., Bi conjugate Chem. 1998, 9, 64-71. Also, a nitrogen mustard-hairpin pyrroleimidazole polyamide conjugate alkylates the directed sites of DNA from the minor groove. Wurtz et al., Chem. Biol. 2000, 7, 153-161. The in situ activation of mitomycin C or aflatoxin B1 by enzymatic reduction or oxidation alkylates duplex DNA at the 5′-CG or G sites, respectively. Tomasz et al., Chem. Biol. 1995, 2, 575-579; Mao et al., Biochemistry 1998, 37, 4374-4387; Bailey et al., Proc. Natl. Acad. Sci. U.S.A. 1996, 93, 1535-1539. Moreover, target-activated CC-1065, duocarmycin and ecteinascidin derivatives alkylate duplex DNA specifically, and are stable in the absence of duplex DNA. Warpehoski et al., J. Am. Chem. Soc. 1995, 117, 295 1-2952; Boger et al., J. Am. Chem. Soc. 1993, 115, 9025-9036; Zewail-Foote et al., J. Med. Chem. 1999, 42, 2493-2497; Pommier et al., Biochemistry 1996, 35, 13303-13309. Such duplex DNA-activated alkylations are attributed to the binding of the drugs in the minor groove of DNA. Boger et al., Acc. Chem. Res. 1999, 32, 1043-1052; Moore et al., J. Am. Chem. Soc. 1998, 120, 2490-2491; Zewail-Foote et al., J. Am. Chem. Soc. 2001, 123, 6485-6495. However, since these target DNA-activations are dependent with respect to the structures of the alkylating agents, the extension of the processes to other alkylating agents for the development of potential target-activated agents is not feasible.

Moreover, in the covalent modification of biological targets, it has been shown that additional selectivity may be obtained by combining the intrinsic chemical reactivity of an agent with a site-directing ligand to form an affinity reagent. Knorre et al., Affinity Modification of Biopolymers 1989 (CRC Press, Boca Raton); Knorre et al., Design and Targeted Reactions of Oligonucleotide Derivatives 1994 (CRC Press, Boca Raton). This approach has been applied with great success when studying proteins and nucleic acids in vitro. However, applications to more complex systems are often stymied by unavoidable side reactions caused by the spontaneous reactivity of the agent and an abundance of nonspecific competitors that overwhelm the potential kinetic advantage of affinity-based localization. Yet, an even higher level of specificity has been obtained with mechanism-based reagents for targets with catalytic activity. Silverman, R. B., Mechanism Based Enzyme Inactivation: Chemistry and Enzymology 1988 (CRC Press, Boca Raton). This strategy relies on the binding and catalytic properties of the target to localize and activate the reagent's inducible reactivity. Paradoxically, the ultimate specificity may be attained by avoiding covalent reactivity altogether and instead designing a species with a slow rate of dissociation from its target so as to approach the physiological equivalent of covalent modification. This objective is now frequently achieved with transition-state analogues and often serves as an initial step in drug development. Wolfenden et al., Curr. Opin. in Struct. Biol. 1991, 1, 780-787.

Nevertheless, the reliance on covalent reactivity remains widespread out of necessity when targets, in particular DNA, lack a predictable catalytic activity. Such suboptimal approaches are perhaps most pertinent to the treatment of life-threatening diseases such as cancer for which few alternatives are available. For example, highly toxic and nonspecific alkylating agents of DNA, such as the N-mustards chlorambucil and cyclophosphamide, are still frequently prescribed against various forms of cancer. Chabner et al., in Goodman and Gilman's The Pharmacological Basis of Therapeutics—10th ed., 2001, 1389-1459 (eds. Hardman, J. G., Limbird, L. E. & Gilman, A. G.) (McGraw-Hill, New York). Only a few covalently reactive drugs such as bleomycin and mitomycin gain some degree of specificity by their need for cellular induction and ability to form preassociation complexes with duplex DNA. Tomasz, M., Chem. Biol. 1995, 2, 575-579; Stubbe et al., Acc. Chem. Res. 1996, 29, 322-330. Still, sequence specificity remains minimal.

The DNA-dependent activation of a bound substrate or ligand is extremely rare and consequently highly prized. Wolkenberg et al., Chem. Rev. 2002, 102, 2477-2495. The most common manner in which DNA can induce or accelerate reactions is through its ability to act as a template and localize reactants. For example, a single strand of DNA can selectively bind and orient two smaller complementary strands to direct their respective reactive appendages. Ma et al., Proc. Natl. Acad. Sci. USA 2000, 97, 11159-11163; Sando et al., J. Am. Chem. Soc. 2002, 124, 2096-2097; Calderone et al., Angew. Chem. Int. Ed. 2002, 41, 4104-4108; Gartner et al., Angew. Chem. Int. Ed. 2003, 42, 1370-1375. Additionally, single- and double-stranded DNA are capable of catalyzing polymerization or ligation of complementary sequences through duplex and triplex recognition and assembly. Rohatgi et al., J. Am. Chem. Soc. 1996, 118, 3332-3339; Li et al., J. Am. Chem. Soc. 2002, 124, 746-747; Li et al., Chem. Biol. 1997, 209-214. However, rate acceleration may also exceed that easily explained solely by the template effects of DNA. In such cases, the conformation and surface properties of nucleic acids likely contribute to the rate enhancements. Geacintov et al., Biochemistry 1982, 21, 1864-1869; Fernando et al., Chem. Res. Toxicol. 1996, 9, 1391-1402; Lamm et al., J. Am. Chem. Soc. 1996, 118, 3326-3331; Shaw et al., J. Am. Chem. Soc. 1991, 113, 7765-7766; Wilson et al., Nature 1995, 374, 777-782; Nagatsugi et al., J. Am. Chem. Soc. 1999, 121, 6753-6754; Lee et al., Chem. Commun. 2002, 2112-2113. A well studied example of this type of DNA-promoted process involves its reaction with cyclopropylpyrroloindole derivatives such as those found in natural products CC-1065, the duocarmycins and bizelsin, currently undergoing clinical investigation. Lin et al., Biochemistry 1991, 30, 3597-3602; Warpehoski et al., J. Am. Chem. Soc. 1995, 117, 2951-2952; Boger et al., Acc. Chem. Res. 1999, 32, 1043-1052; Pitot et al., Clinical Cancer Res. 2002, 8, 712-717. Unfortunately, whether such processes may be translated more generally to other molecular structures remains unclear.

It is therefore a purpose of the present invention to develop new target-activating mechanisms for the selective, target-activated modification of target molecules. It is a purpose of the present invention to develop new methods and reagents for the selective, target-activated modification of target molecules. Such development is expected to lead to general principles that can be applied for effective drug design and medical treatment.

### SUMMARY OF THE INVENTION

The present invention relates to methods for selective, target-activated modification of target molecules. The present invention further relates to reagents and intermediates in the target-activated alkylation of target molecules.

The present invention therefore provides for methods for the selective, target-activated modification of a target molecule, said method comprising: providing a reagent comprising (i) a ligand including at least one recognition element and (ii) at least one reactive component, wherein the reagent is capable of reversible, intramolecular attachment between the ligand and the reactive component; specifically binding the reagent with the target molecule via the at least one recognition element of the ligand; and binding the reactive component of the reagent to the target molecule.

The present invention also provides for reagents for the selective, target-activated modification of a target molecule, said reagent comprising (i) a ligand including at least one recognition element and (ii) at least one reactive component, the reagent capable of reversible, intramolecular attachment between the ligand and the reactive component; wherein the reagent is capable of specifically binding with the target molecule via the at least one recognition element of the ligand; and wherein the reagent is capable of binding the reactive component of the reagent to the target molecule.

The present invention further provides for intermediates in the selective, target-activated modification of a target molecule, said intermediate comprising the target molecule and a reagent comprising (i) a ligand including at least one recognition element and (ii) at least one reactive component, the reagent capable of reversible, intramolecular attachment between the ligand and the reactive component; wherein the reagent is specifically bound with the target molecule via the at least one recognition element of the ligand; and wherein the reagent is capable of binding the reactive component to the target molecule.

### BRIEF DESCRIPTION OF THE DRAWINGS

FIG. 1 is an illustration of general principles of selective, target-activated modification in accordance with preferred embodiments of the present invention.

FIG. 2 shows HPLC detection of formation of quinone methide-DNA (“QM-DNA”) self-adduct 20 after desilylation of compound 17 in the presence of KF and absence of complementary DNA. Reaction was initiated by adding KF (1.0 M) to 17 (2.5 μM) in MES (pH 7.0, 20 mM) at 25° C.

FIG. 3 shows desilyation of t-butyldimethylsilyl-acetate-oligonucleotide 1 (“TBDMS-acetate-OD1”) compound 17 in the presence of KF and an absence of complementary DNA. Reaction was initiated by adding KF (1.0 M) to compound 17 (2.5 μM) in MES (pH 7.0, 20 mM) at 25° C. and progress of the reaction was monitored by HPLC analysis.

FIG. 4 shows consumption of acetate-phenol conjugate 18 in the absence of complementary DNA over time. Reaction was initiated by adding KF (1.0 M) to compound 17 (2.5 μM) in MES (pH 7.0, 20 mM) at 25° C. and progress of the reaction was monitored by HPLC analysis. The amount of conjugate 18 was determined in relation to the initial amount of compound 17 in the reaction.

FIG. 5 show alkylation of target diphenyl-oligonucleotide 2 (“diphenyl-OD2”) by QM-DNA self-adduct 20 over time. QM-DNA self-adduct 20 was formed by treating compound 17 (5.0 μM) with KF (1.0 M) in MES (pH 7.0, 20 mM) at 25° C. for 40 h. Alkylation was initiated by adding self-adduct 20 (2.5 μm) to diphenyl-OD2 (2.5 μM) in MES (pH 7.0, 20 mM) and KF (1.0 M) and the progress of reaction at 25° C. was monitored by HPLC analysis.

FIG. 6 shows selective alkylation by QM-DNA self-adduct 20 detected by gel electrophoresis. QM-DNA self-adduct 20 was formed by treating compound 17 (2.2 μM) with KF (1.0 M) in MES (pH 7.0, 20 mM) at 25° C. for 8 days. Alkylation was initiated by adding self-adduct 20 (1.1 μM) to ³²P-OD2 or ³²P-OD3 (1.0 μM) in MES (pH 7.0, 20 mM) and KF (1.0 M). Alkylation yield was calculated in relation to the total amount radiolabeled.

FIG. 7 shows selective alkylation of oligonucleotide 2 (“OD2”) by QM-DNA self-adduct 20 in the presence of DNA competitors. QM-DNA self-adduct 20 was formed by treating compound 17 (2.2 μM) with KF (1.0 M) in MES (pH 7.0, 20 MM) at 25° C. for 8 days. Alkylation was initiated by adding self-adduct 20 (1.1 μM) to ³²P-OD2 (1.0 μM) and DNA competitors in MES (pH 7.0, 20 mM) and KF (1.0 M) except lane 1 used as a control. DNA competitors: lanes 1-2, none; lane 3, OD3 (10 μM); lane 4, duplex calf thymus DNA (10.0 μM); lane 5, denatured calf thymus DNA (10.0 μM). Alkylation yield was calculated related to the total amount radiolabeled.

FIG. 8 shows selective alkylation of OD2 by QM-DNA self-adduct 20 in the presence of thiols. QM-DNA self-adduct 20 was formed by treating compound 17 (2.2 μM) with KF (1.0 M) in MES (pH 7.0, 20 mM) at 25° C. for 8 days. Alkylation was initiated by adding self-adduct 20 (1.1 μM) to ³²P-OD2 (1.0 μM) and thiols in MES (pH 7.0, 2.0 mM) and KF (1.0 M) except lane 1 used as a control. Thiols: lanes 1-2, none; lane 3, 45 μM; lane 4, 450 μM. Alkylation yield was calculated in relation to the total amount radiolabeled.

FIG. 9 shows time dependent target alkylation of OD2 with QM-DNA self-adduct 20 detected by gel analysis. Desilyation was initiated by adding KF (1.0 M) to compound 17 (2.2 μM) in MES (pH 7.0, 20 mM) and left incubated at 25° C. for 0-8 days. Alkylation of ³²P-OD2 was initiated by adding the resulting solution (1.1 μM) to ³²P-OD2 (1.0 μM) in MES (pH 7.0, 20 mM) and KF (1.0 M) and left at ambient temperature for 8 days. Percent alkylation was calculated in relation to the total amount radiolabeled.

FIG. 10A shows the desilylation of TBDMS-acetate-OD1 (17) in the presence of KF and complementary diphenyl-OD2. Reaction was initiated by adding KF (1.0 M) to compound 17 (2.5 μM) and diphenyl-OD2 (2.5 μM) in MES (pH 7.0, 20 mM) at 25° C. and progress of the reaction was monitored by HPLC analysis.

FIG. 10B shows the consumption of acetate-phenol conjugate 18 in the presence of complementary diphenyl-OD2 over time. Reaction was initiated by adding KF (1.0 M) to compound 17 (2.5 μM) and diphenyl-OD2 (2.5 μM) in MES (pH 7.0, 20 mM) at 25° C. and progress of the reaction was monitored by HPLC analysis. The amount of conjugate 18 was determined in relation to the initial amount of compound 17 in the reaction.

FIG. 11A shows time dependent target alkylation of OD2 with QM-DNA self-adduct 20 detected by gel analysis. Desilylation was initiated by adding KF (1.0 M) to compound 17 (2.2 μM) in MES (pH 7.0, 20 mM) and left incubated at 25° C. for 0-8 days. Alkylation of ³²P-OD2 was initiated by adding the resulting solution (1.1 μM) to ³²P-OD2 (1.0 μM) in MES (pH 7.0, 20 mM) and KF (1.0 M) and left at ambient temperature for 8 days. Percent alkylation was calculated in relation to the total amount radiolabeled.

FIG. 11B shows the desilylation of compound 17 and subsequent target alkylation in the presence of thiol. Alkylation was initiated by adding self-adduct 20 (1.1 μM) with/without thiol to ³²P-OD2 (1.0 μM) in MES (pH 7.0, 20 mM) and KF (1.0 M) and left at ambient temperature for 8 days except lane 1 as a control. Lane 2: QM-DNA self-adduct 20 was formed by treating compound 17 (2.2 μM) with KF (1.0 M) in MES (pH 7.0, 20 mM) for 8 days; lane 3: QM-DNA self-adduct 20 was formed by treating compound 17 (2.2 μM) with KF (1.0 M) in MES (pH 7.0, 20 mM) and thiol (0.99 mM) for 8 days; lane 4: desilylation was initiated by adding KF (1.0 M) to compound 17 (2.2 μM) in MES (pH 7.0, 20 mM) and left incubated at 25° C. for 2 days and then thiol (0.99 mM) was added; the resulting solution was left at 25° C. for 6 days. The percentage conversion was calculated based on the total amount of radiolabeled OD2. DNA cleavage in the background was due to the oxidation of thiols.

FIG. 12A shows the alkylation of target diphenyl-OD2 by QM-DNA self-adduct 20 in the presence of thiols. QM-DNA self-adduct 20 was formed by treating compound 17 (5.0 μM) with KF (1.0 M) in MES (pH 7.0, 20 mM) at 25° C. for 8 days. Alkylation was initiated by adding self-adduct 20 (2.5 μM) to diphenyl-OD2 (2.5 μM) and mercaptoethanol (1.125 mM) in MES (pH 7.0, 20 mM) and KF (1.0 M) and the progress of reaction at 25° C. was monitored by HPLC analysis

FIG. 12B shows an ESI-MS of an oligonucleotide.

FIG. 13 shows an ESI-MS of oligonucleotide intermediates and products.

FIG. 14 shows an ESI-MS of oligonucleotide intermediates and products.

FIG. 15 shows an ESI-MS of oligonucleotide intermediates and products, including the aklylation product of OD1-QMP self-adduct 20 and diphenyl-OD2.

FIG. 16 illustrates self-adduct formation from OD1-QMP and its subsequent target alkylation in the presence of a thiol. ³²P-OD2 (1.0 μM, lane 1) was added to initiate target alkylation (8 days ambient temperature) with the various preparations of OD1-QMP in 20 mM MES pH 7. Lane 2: OD1-QMP (1.1 μM) was pre-incubated with 1.0 M KF in 20 mM MES pH 7 for 8 days under ambient temperature. Lane 3: OD1-QMP (1.1 μM), β-mercaptoethanol (1 mM) and KF (1 M) were treated with OD2 directly. Lane 4: OD1-QMP (1.1 μM) was pre-incubated with 1.0 M KF in 20 mM MES pH 7 for 2 days under ambient temperature. Incubation was then continued for another 6 days in the presence of β-mercaptoethanol (1 mM).

FIG. 17 illustrates reversible formation of an intramolecular adduct and intermolecular transfer to a target molecule, in accordance with preferred embodiments of the present invention.

FIG. 18 illustrates oligonucleotides according to the present invention.

FIG. 19A illustrates formation of an intramolecular self-adduct, in accordance with preferred embodiments of the present invention.

FIG. 19B illustrates alkylation of a target molecule by an intramolecular self-adduct, in accordance with preferred embodiments of the present invention.

FIG. 20 illustrates added base pairing driving intermolecular aklylation of a target, in accordance with preferred embodiments of the present invention.

### DETAILED DESCRIPTION OF THE INVENTION

The present invention concerns methods and reagents for the selective, target-activated modification of target molecules, preferably biopolymers, such as nucleic acids or protein. The present invention further concerns target-activating mechanisms that can be applied as general principles for effective drug design and medical research and treatment.

For example, in a preferred embodiment, the present invention concerns methods for selective, target-activated modification of a target molecule, said method comprising: providing a reagent comprising (i) a ligand including at least one recognition element and (ii) at least one reactive component, wherein the reagent is capable of reversible, intramolecular attachment between the ligand and the reactive component; specifically binding the reagent with the target molecule via the at least one recognition element of the ligand; and binding the reactive component of the reagent to the target molecule.

In another preferred embodiment, the present invention concerns reagents for the selective, target-activated modification of a target molecule, said reagent comprising (i) a ligand including at least one recognition element and (ii) at least one reactive component, the reagent capable of reversible, intramolecular attachment between the ligand and the reactive component; wherein the reagent is capable of specifically binding with the target molecule via the at least one recognition element of the ligand; and wherein the reagent is capable of binding the reactive component of the reagent to the target molecule.

Also in a preferred embodiment, the present invention concerns intermediates in the selective, target-activated modification of a target molecule, said intermediate comprising the target molecule and a reagent comprising (i) a ligand including at least one recognition element and (ii) at least one reactive component, the reagent capable of reversible, intramolecular attachment between the ligand and the reactive component; wherein the reagent is specifically bound with the target molecule via the at least one recognition element of the ligand; and wherein the reagent is capable of binding the reactive component to the target molecule.

As demonstrated throughout the application, the inventors of the present invention have investigated target-activated DNA alkylation by quinone methide (“QM”), with delivery via oligonucleotide or DNA conjugates. Although at the outset the mechanism of activation was not entirely clear, Li et al., Bioconjugate Chem. 1994, 5, 497-500; Li et al., J. Am. Chem. Soc. 1991, 113, 7771-7773, several possible mechanisms have been investigated, including DNA-catalyzed cleavage, Roth et al., Proc. Natl. Acad. Sci. U.S.A. 1998, 95, 6027-6031; Breaker et al., Chem. Biol. 1994, 1, 223-229, DNA-hybridization directed alkylation, Gartner et al., J. Am. Chem. Soc. 2001, 123, 6961-6963; Ma et al., Proc. Natl. Acad. Sci. U.S.A. 2000, 97, 11159-11163; Mao et al., Proc. Natl. Acad. Sci. U.S.A. 1999, 96, 66 15-6620; Shaw et al., J. Am. Chem. Soc. 1991, 113, 7765-7766, and the activation of reversible QM nucleobase self-adducts. Veldhuyzen et al., J. Am. Chem. Soc. 2001, 123, 11126-11132.

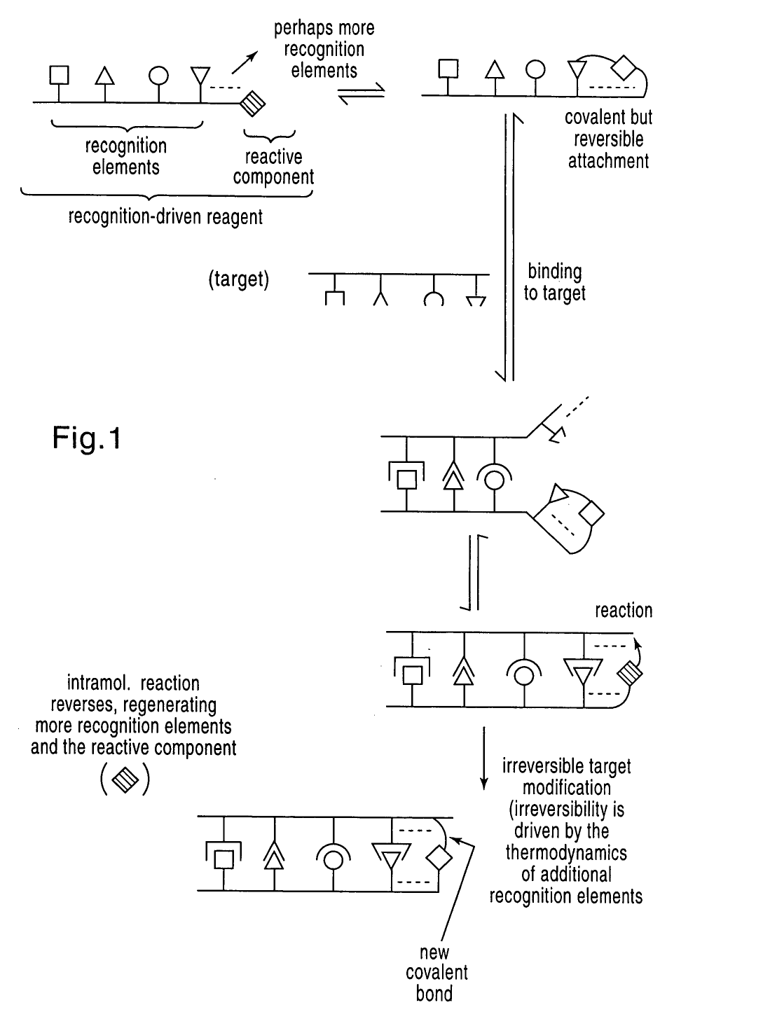

It has now been discovered that the thermodynamics of target recognition is the driving force for activating latent QM derivatives for the alkylation of target molecules. That is, with reference to FIG. 1, which shows general principles of selective, target-activated modification, it can be seen that target-activated modification may be possible because of the (1) reversible formation of intramolecular self-adducts between an electrophilic appendage (reactive component) and an attached ligand (including recognition elements), and (2) intermolecular transfer of the appendage to the target in a manner dependent on its recognition. The significance of the resulting target-activated mechanism is that it may be extended as a general strategy for the development of various target-activated drugs for any target (e.g., cellular target) by coupling electrophilic alkylating moieties with target-recognizing agents. Accordingly, any de novo target-activated agents (e.g., alkylating agents) that act reversibly may be used by following the general principles of the present invention, thus allowing for effective drug design and medical research.

FIG. 1 illustrates a preferred method and reagent for the selective, target-activated modification of a target molecule, the method comprising the step of providing a reagent comprising (i) a ligand including at least one recognition element and (ii) at least one reactive component, wherein the reagent may be capable of reversible, intramolecular attachment between the ligand and the reactive component. The intramolecular attachment between the ligand and the reactive component may occur through a variety of binding forces well known to those of ordinary skill in the art including, but not limited to, covalent, hydrogen, van der Waals and ionic forces. The intramolecular attachment between the ligand and the reactive component may preferably occur through a reversible, covalent bond. Without being bound by scientific theory, it is believed that the attachment between the ligand and reactive component inhibits competing intermolecular reactions with the reactive component. Where the ligand and target are nucleic acid, the reagent may be stable in the absence of complementary sequences of nucleic acid and/or the presence of philes, water and cellular nucleophiles.

The method may also comprise the step of specifically binding the reagent with the target molecule via the at least one recognition element of the ligand. The reagent may be capable of specific binding with the target molecule and/or reversible, intramolecular attachment between the ligand and the reactive component. Where the ligand and target are nucleic acid, specific binding may occur between complementary nucleic acid sequences. Specific binding may be via duplex or triplex hybridization. Where the ligand and target are nucleic acid, complementary base pairing may induce a conformational change to promote binding of the reactive component of the reagent to the target molecule.

The method may also comprise the step of binding the reactive component of the reagent to the target molecule. The binding of reactive component and target molecule may occur through a variety of binding forces well known to those of ordinary skill in the art including, but not limited to, covalent, hydrogen, van der Waals and ionic forces. Binding of the reactive component and target molecule may preferably be via a reversible, covalent bond. The reactive component may alkylate the target molecule.

FIG. 1 also illustrates an intermediate comprising a target molecule and a reagent comprising (i) a ligand including at least one recognition element and (ii) at least one reactive component, wherein the reagent may be capable of reversible, intramolecular attachment between the ligand and the reactive component. The reagent may be specifically bound with the target molecule via the at least one recognition element of the ligand. The reagent may also be capable of binding the reactive component of the recognition-driven reagent to the target molecule.

The intermediate is capable of intramolecular attachment between the ligand and the reactive component, that may occur through a variety of binding forces well known to those of ordinary skill in the art including, but not limited to, covalent, hydrogen, van der Waals and ionic forces. The intramolecular attachment between the ligand and the reactive component may preferably occur through a reversible, covalent bond. Where the ligand and target are nucleic acid, the reagent may be stable in the absence of complementary sequences of nucleic acid.

The intermediate may be capable of specific binding with the target molecule and/or reversible, intramolecular attachment between the ligand and the reactive component. Where the ligand and target are nucleic acid, specific binding may occur between complementary nucleic acid sequences. Specific binding may occur by duplex or triplex hybridization. Where the ligand and target are nucleic acid, complementary base pairing may induce a conformational change to promote binding of the reactive component of the reagent to the target molecule.

The intermediate may be capable of binding the reactive component and target molecule via a variety of binding forces well known to those of ordinary skill in the art including, but not limited to, covalent, hydrogen, van der Waals and ionic forces. Binding of the reactive component and target molecule may preferably be via a reversible, covalent bond. The reactive component may alkylate the target molecule.

Those of ordinary skill in the art will recognize that a variety of reactive components are suitable for use with the present invention. Such reactive components may include, but are not limited to, imino-quinone methide and quinone methide, as demonstrated throughout the application. Perhaps more important, those of ordinary skill in the art will recognize that through application of the general principles of the present invention, a wide variety of reactive components will be suitable for use in the present invention.

With further reference to FIG. 1, it is pointed out that those of ordinary skill in the art will recognize that a reagent may preferably be capable of reversibly, covalently binding the reactive component of the recognition-driven reagent to the target molecule. In addition, the intramolecular attachment between ligand and reactive component may preferably be via a reversible, covalent bond. However, such intramolecular and intermolecular binding may occur via other binding forces well known to those of ordinary skill in the art including, but not limited to, hydrogen, van der Waals and ionic forces.

Those of ordinary skill in the art will also recognize that specific binding may occur via hybridization. For example, specific binding may occur via duplex or triplex hybridization. Where the ligand and target are nucleic acid, specific binding may occur between complementary nucleic acid sequences. Where the ligand and target are nucleic acid, complementary base pairing may induce a conformational change to promote binding of the reactive component of the reagent to the target molecule.

The present invention is suitable for use with nucleic acid and protein, although it will be recognized that a variety of other reagents and targets can be used in the present invention. For instance, those of ordinary skill in the art will recognize a variety of commercially available ligands known in the art, for example standard analogues (e.g., synthetic) of oligonucleotides. It is noted that a ligand of the present invention may comprise nucleic acid. Indeed, ligands may include, but are not limited to, single and double stranded DNA, RNA, oligonucleotides, oligonucleotide derivates and analogues, peptide nucleic acid (“PNA”), link nucleic acid (“LNA”) and methylphosphonates. Nucleic acid may be selected from the group consisting of single and double stranded DNA and RNA. Nucleic acid may be selected from the group consisting of oligonucleotides, oligonucleotide derivates, peptide nucleic acid (“PNA”), link nucleic acid (“LNA”) and methylphosphonates.

The at least one recognition element of the present invention may be suitable for use with nucleic acid and protein, as well as other target molecules. For example, where the target is nucleic acid, the at least one recognition element of the ligand may comprise a nucleotide. The at least one recognition element of the ligand may include, but is not limited to, ribonucleotides and deoxyribonucleotides. Also for example, where the target molecule is protein, those of ordinary skill in the art will recognize a variety of available recognition elements, for instance synthetic analogues of peptides or peptido-mimentics. Recognition elements may also include, but are not limited to, amino acids, amino acid analogues, peptides, analogue peptides, protein antagonists and agonists, transition state analogues and substrate analogues.

The present invention is suitable for targeting a variety of target molecules including, but not limited to, nucleic acid and protein. For example, target molecules may comprise amino acids. Target molecules may comprise nucleic acid. Target molecules may include, but are not limited to, single and double stranded DNA, RNA, oligonucleotides, oligonucleotide derivates, PNA, LNA and methylphosphonates. The nucleic acid may be selected from the group consisting of DNA and RNA.

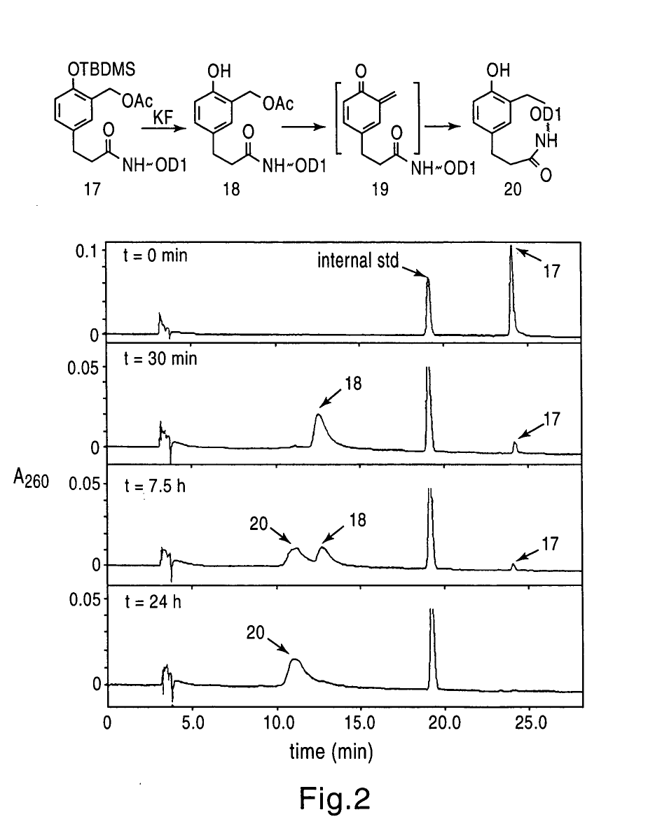

With reference to the drawings including FIGS. 1 and 2, it can be seen that a variety of compounds may be suitable for use with the present invention, for example QM-oligonucleotide self-adducts having the general formula 20. The intramolecular self-adduct 20 may be formed from a QM-oligonucleotide conjugate. The self-adduct is stable in the absence of a complementary sequence of DNA, yet remarkably is able to alkylate target DNA upon duplex or triplex hybridization. The self-adduct has been shown to be stable in the absence of a complementary sequence of DNA for at least 8 days. Neither formation of the self-adduct nor transfer of QM to the target is significantly quenched by 450-fold excess β-mercaptoethanol. Similarly, non-complementary DNA is neither subject to alkylation by the self-adduct nor able to effect its consumption. Thus, it appears that reversible trapping of the QM through an intramolecular reaction is enough to inhibit competing intermolecular reactions. Complementary base pairing induces the conformational change necessary to promote intermolecular transfer of QM.

Target-activated alkylation of DNA has been successfully achieved by reversible QMoligonucleotide self-adducts of general formula 20, through a hybridization-dependent process. The intra-strand QM-oligonucleotide self-adduct of general formula 20 is preferably formed in the absence of complementary DNA. Alkylation by the self-adduct of general formula 20 is complementary-sequence selective, and therefore, is a hybridization induced process. The ability of the self-adduct of general formula 20 to alkylate a complementary target may be persistent for at least 8 days and is not quenched by an excess amount of DNA competitors and mercaptoethanol.

In the absence of complementary DNA, reversible, irreversible and latent self-adducts may be formed from QM. Reversible self-adducts regenerate QM and convert to irreversible self-adducts as a stable product Remarkably, latent self-adducts remain stable in the absence of complementary target, yet are able to regenerate QM upon hybridization with a target molecule, and even alkylate the target.

It will be realized that the present invention may perform a variety of functions, including use as a diagnostic and/or a therapeutic. For example, the present invention may function to specifically bind a probe (e.g., diagnostic) to a DNA or RNA sequence. The present invention may function to identify a DNA or RNA sequence by binding a probe to such DNA or RNA sequence. The present invention may also function in the site-specific delivery of drugs, for example to nucleic acid or amino acid sequences. In addition, the present invention may be used to modify a gene target so as to inactivate or shut down the gene, even modify RNA so as to inhibit protein production. The present invention may further be used to modify a protein or enzyme target, for instance in the inactivation of such protein or enzyme. It is to be realized that those of ordinary skill in the art will recognize a variety of other functions suitable for the present invention.

Generalization of the approach used with the self-adducts of the invention, based on the reversible intramolecular trapping of a reactive component by a ligand with at least one recognition element, is applicable to wide-ranging applications in targeting nucleic acids, proteins and other targets. It is significant that studies with QM have demonstrated the hybridization induced activation of latent self-adducts to regenerate QM and subsequently alkylate a target. This process is driven by target-activation to achieve a thermodynamically stable product, and may be utilized as a general strategy to develop selective, target-activated agents, for example by coupling latent electrophiles of reactive components with target recognition elements.

### EXAMPLES

### Materials and Methods

All organic solvents and reagents obtained commercially were used without purification unless noted otherwise. Oligonucleotides were purified by polyacrylamide gel electrophoresis under standard conditions. Oligonucleotide conjugates, self adducts and interstrand alkylation products were also separated with a reverse-phase C-18 column (Varian Microsorb-MV, 300 □ pore 250 mm) with a gradient (30 min, 1 mL/min) of 10-55% acetonitrile in aqueous triethylammonium acetate (50 mM, pH 5.0) as controlled by a JASCO PU-980 HPLC system (Easton, Md.). Samples (20 to 150 pmol DNA) were manually collected during elution from the HPLC and analyzed by electron spray ionization mass-spectroscopy (ESI-MS, Thermo Finnigan LCQ) using direct injection with a voltage of −3.6 kV and a capillary temperature at 170° C.

### Example 1

Formation of a QM-oligonucleotide self-adduct. Recent appreciation for the reversible nature of QM alkylation, Modica et al., J. Org. Chem. 2001, 66, 41-52; Veldhuyzen et al., J. Am. Chem. Soc. 2001,123, 11126-11132, suggests that transient self-adducts of QM with DNA might function as an effective delivery agent for this highly reactive intermediate, for example as the malondialdehyde adduct of deoxyguanosine (dG) efficiently transports malondialdehyde in vivo. Dedon et al., Proc. Natl. Acad. Sci. USA 1998, 95, 11113-11116; Plastaras et al., Chem. Res. Toxicol. 2000, 13, 1235-1242. Our laboratory has traditionally relied on fluoride dependent deprotection of O-silylated precursors (QMP) and their site-directed conjugates for inducible alkylation by ortho-benzoquinone methide. Veldhuyzen et al., J. Am. Chem. Soc. 2001, 123, 11126-11132; Li et al., J. Am. Chem. Soc. 1991, 113, 7771-7773; Zeng et al., J. Org. Chem. 1996, 61, 9080-9081; Pande et al., J. Am. Chem. Soc. 1999, 121, 6773-6779; Veldhuyzen et al., Chem. Res. Toxicol. 2001, 14, 1345-1351; Zhou et al., Bioorg. Med. Chem. 2001, 9, 2347-2354. However, studies have revealed a novel fluoride independent activation of oligonucleotide-QMP conjugates. Li et al., J. Am. Chem. Soc. 1991, 113, 7771-7773.

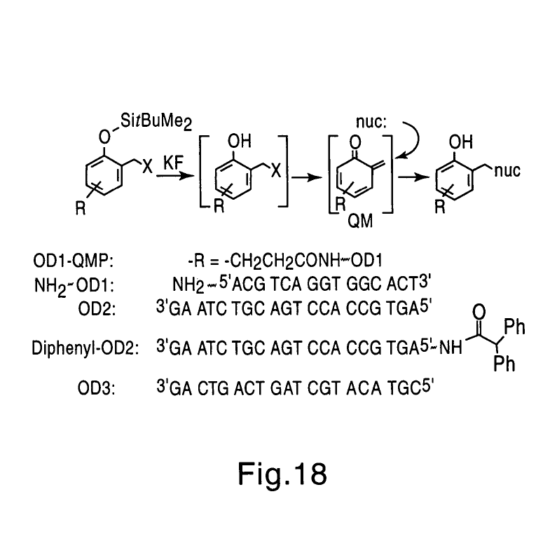

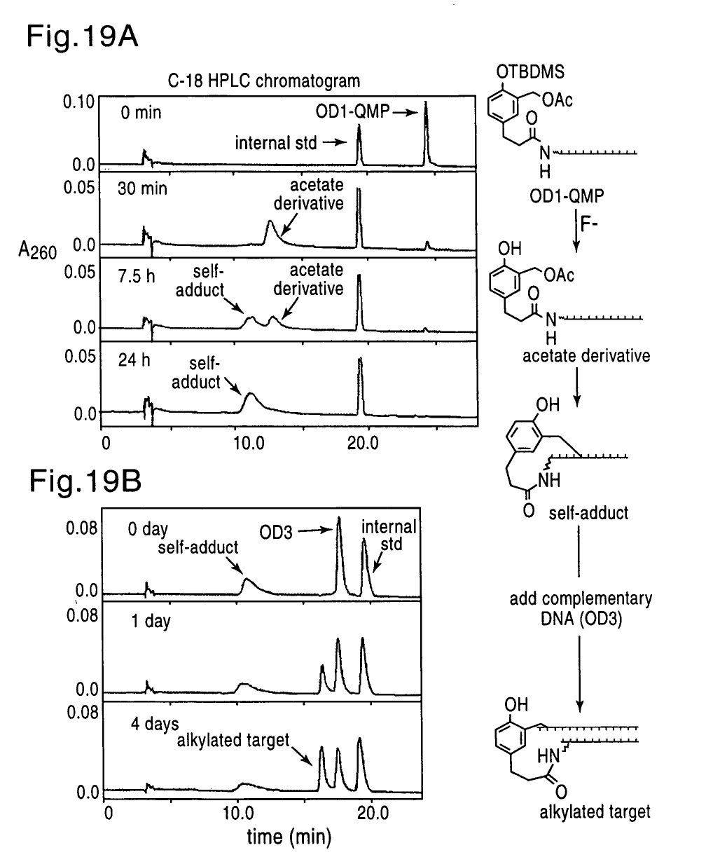

With reference to FIG. 18, it is noted that such surprising observations have now been explained by formation of a self-adduct that is capable of intermolecular and target promoted QM transfer. The possibility of target-activated reactions based on QM chemistry originate from at least two observations: (1) formation of QM-oligonucleotide self-adducts; and (2) alkylation of complementary strands of DNA by the self-adduct. A combination of HPLC and ESI-MS analysis provided the first evidence that OD1-QMP efficiently formed a self-adduct. Treatment of this conjugate with KF rapidly removed the t-butyldimethylsilyl (TBDMS) group and generated the unprotected phenol derivative as evident from its m/z of 1663 ([M-3H⁺]³⁻, calc. 1663). See FIG. 19A. This phenol derivative in turn, spontaneously, but more slowly, eliminated acetate to form a new product with a m/z of 1643 that is consistent with a QM self-adduct ([M-3H⁺]³⁻, calc. 1643). This product was formed in >90% yield from the OD1-QMP parent, assuming no significant change in extinction coefficients. See FIG. 19.

### Example 2

The addition of water to QM is not competitive with intramolecular reactions. The absence of any detectable water adduct formed in competition with the QM self-adduct was initially surprising. Despite the slow rate of addition compared to simple quinone methide, Modica et al., J. Org. Chem. 2001, 66, 41-52, its concentration is typically orders of magnitude greater than other potential nucleophiles. Nevertheless, no m/z of 1649 ([M-3H⁺]³⁻ calc for the water adduct) was observed at any point through the HPLC chromatogram. See FIG. 19. While dehydration of the potential water adduct was not expected during ESI-MS analysis, if there had been dehydration, a similar mass as the self-adduct would have been observed. Consequently, a positive control for the formation and detection of a water adduct was performed.

The only nucleotide that does not readily form an adduct with QM is thymidine. Veldhuyzen et al., J. Am. Chem. Soc. 2001, 123, 11126-11132; Pande et al., J. Am. Chem. Soc. 1999, 121, 6773-6779. Therefore a QMP conjugate containing only thymidine (T₁₀-QMP) was synthesized. Incubation of this conjugate with KF for 24 hrs yielded a single product in a 98% yield as detected by HPLC. ESI-MS of this product was consistent with a water adduct ([M-2H⁺]²⁻ of 1668, calc. 1668 and [M-3H⁺]³⁻ of 1112, calc. 1111) and no self-adduct was evident. Thus, the absence of a water adduct for OD1-QMP reflects a lack of formation rather than an inability to be detected.

### Example 3

Target DNA alkylated by a complementary QM-oligonucleotide self-adduct. HPLC and ESI-MS contributed to the observation regarding alkylation of a DNA strand by its complementary OD1-QMP self-adduct. Since the target oligonucleotide, OD2 (see FIG. 18), co-eluted with the self-adduct using reverse phase (C-18) chromatography, the 5′-terminus of the target was extended with a diphenylacetylated aminolinker (diphenyl-OD2). This modification is distal to the possible sites of reaction and allowed for resolution of all reactants. Within 1 day, a single new species was evident from a stoichiometric mixture of the self-adduct and diphenyl-OD2. See FIG. 19B. This product alone continued to accumulate and both the self adduct and diphenyl-OD2 were consumed over 4 days. The m/z signals detected for the product (1628 and 1900) are consistent with those calculated for an alkylated duplex formed by intermolecular QM transfer ([M-7H⁺]⁷⁻, calc. 1629 and [M-6H⁺]⁶⁻, calc. 1900). Thus, the self-adduct was able to react with a complementary strand covalently even though it would otherwise persist unchanged over the same period in the absence of this target. Again, there was no indication that water was able to quench the OD1-QMP self-adduct.

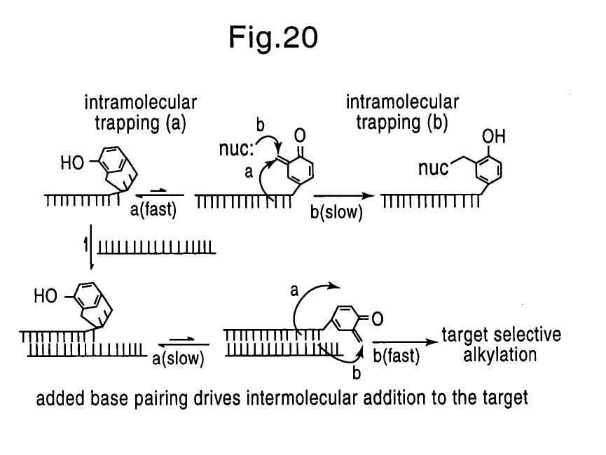

The apparent stability of the self-adduct in the absence of a complementary strand is not incompatible with reactivity in the presence of such a strand. With reference to FIG. 20, it is believed that the self-adduct remains in equilibrium with its high energy QM intermediate since the most reactive nucleophiles of DNA add reversibly to QM. For example, the N1 of dA is both a strong nucleophile and an efficient leaving group. Veldhuyzen et al., J. Am. Chem. Soc. 2001, 123, 11126-11132. Intramolecular addition to reform the self-adduct remains favorable even in the presence of water until hybridization to a complementary strand occurs. Target recognition and duplex formation is still possible from those nucleotides not significantly constrained by the intramolecular QM adduct. See FIG. 20. More important, all constraints are removed once the self-adduct equilibrates back to its QM intermediate. Spontaneous formation of the full complement of base pairs then creates a thermodynamic barrier for reforming the self-adduct (see FIG. 20) and promotes alkylation of the target sequence instead. Hence, a gain in base pairing energy may be used to drive intermolecular transfer of the QM at the expense of an otherwise competitive intramolecular reaction. See FIG. 20.

### Example 4

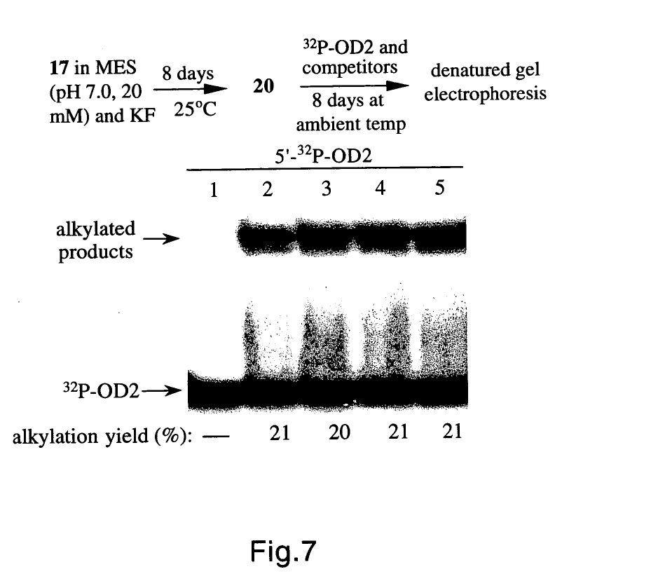

Noncomplementary DNA does not compete for alkylation by QM-oligonucleotide self-adduct. The mechanism of target-activated alkylation proposed in FIG. 20 suggests that alkylation is limited to complementary regions of DNA. This was confirmed by adding a noncomplementary strand, OD3 (see FIG. 18), in 10-fold excess (10 μM) over the self-adduct of OD1-QMP and its complement OD2. Electrophoretic analysis of the alkylation products indicates that OD3 had no appreciable effect on the yield of OD2 alkylation (see FIG. 7, lanes 2 versus 3). A similar lack of competition was observed in the presence of native or denatured calf thymus DNA (see FIG. 7, lanes 4 and 5). Additionally, no alkylation of 5′-[³²P]-OD3 was detected by the self-adduct of OD1-QMP in the absence OD2. The self-adduct thus acts selectively to transfer its QM to a complementary sequence exclusively. Neither water nor nucleophilic sites of noncomplementary DNA effectively quench the transient QM formed by the self-adduct. See FIG. 7.

### Example 5

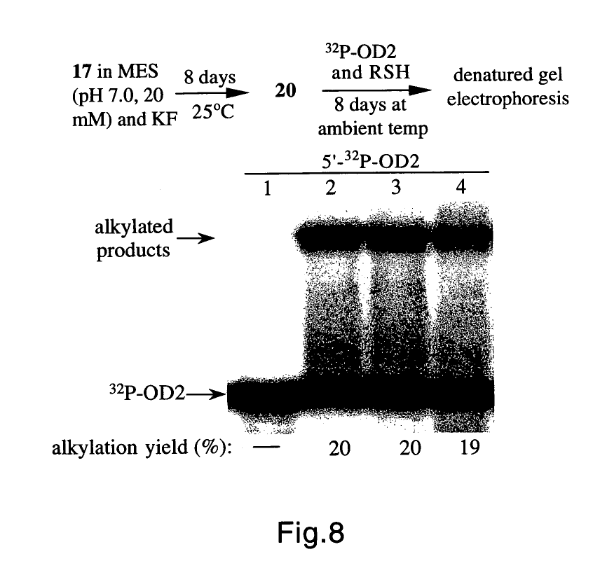

Excess_{.}-Mercaptoethanol does not compete for alkylation by QM-oligonucleotide self-adduct. The inability of solvent to trap the QM intermediate during equilibration with the intramolecular self-adduct may be rationalized by the relatively low nucleophilicity of water, and a similar inability of noncomplementary DNA to trap the QM may be rationalized by electrostatic repulsion of the nucleotide strands. The lack of quenching by 450 equivalents of_{.}-mercaptoethanol is even more remarkable. Alkylation of one equivalent of 5′-[³²P]-OD2 (1 μM) and 1.1 equivalents of the OD1-QMP self adduct was not significantly effected by simultaneous addition of_{.}-mercaptoethanol (0, 45 and 450 μM). See FIG. 8, lanes 2, 3 and 4.

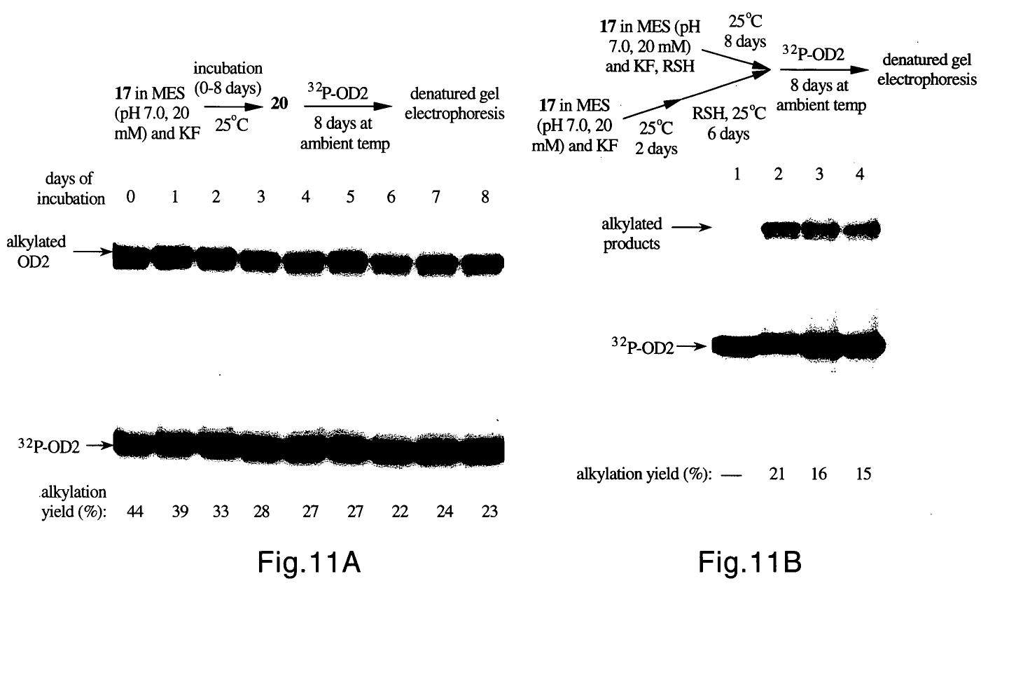

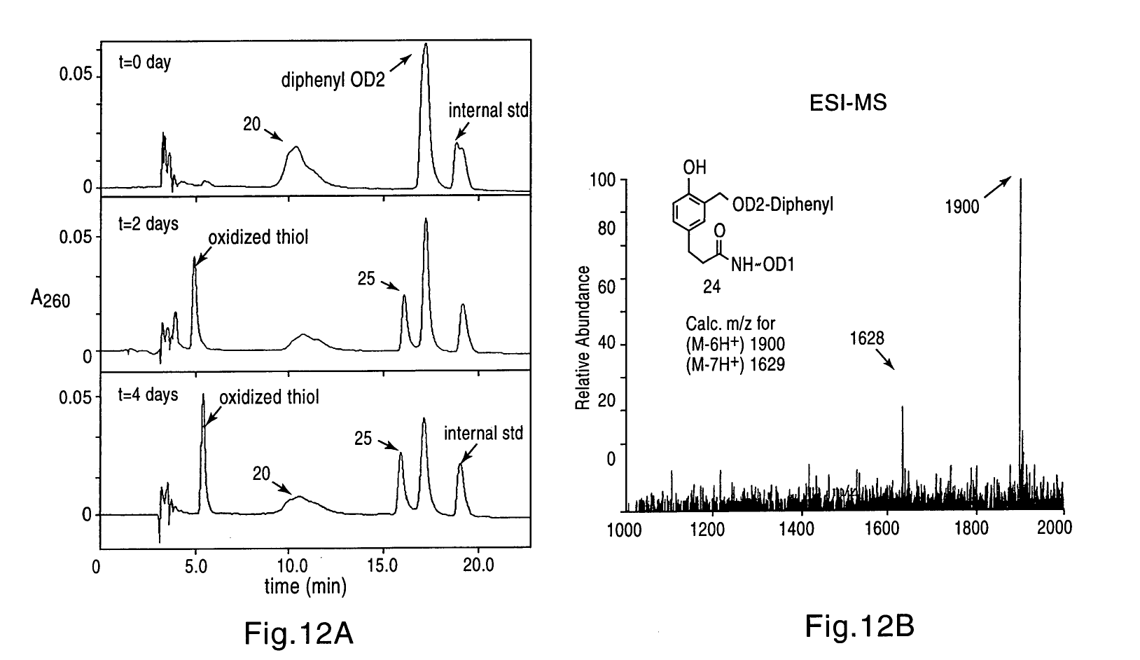

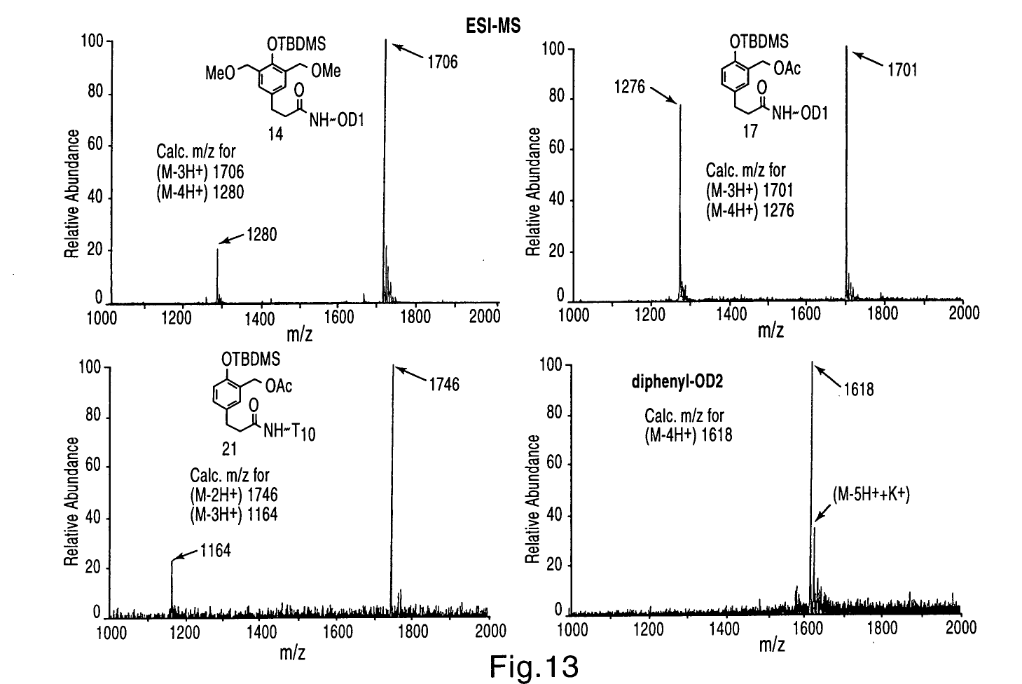

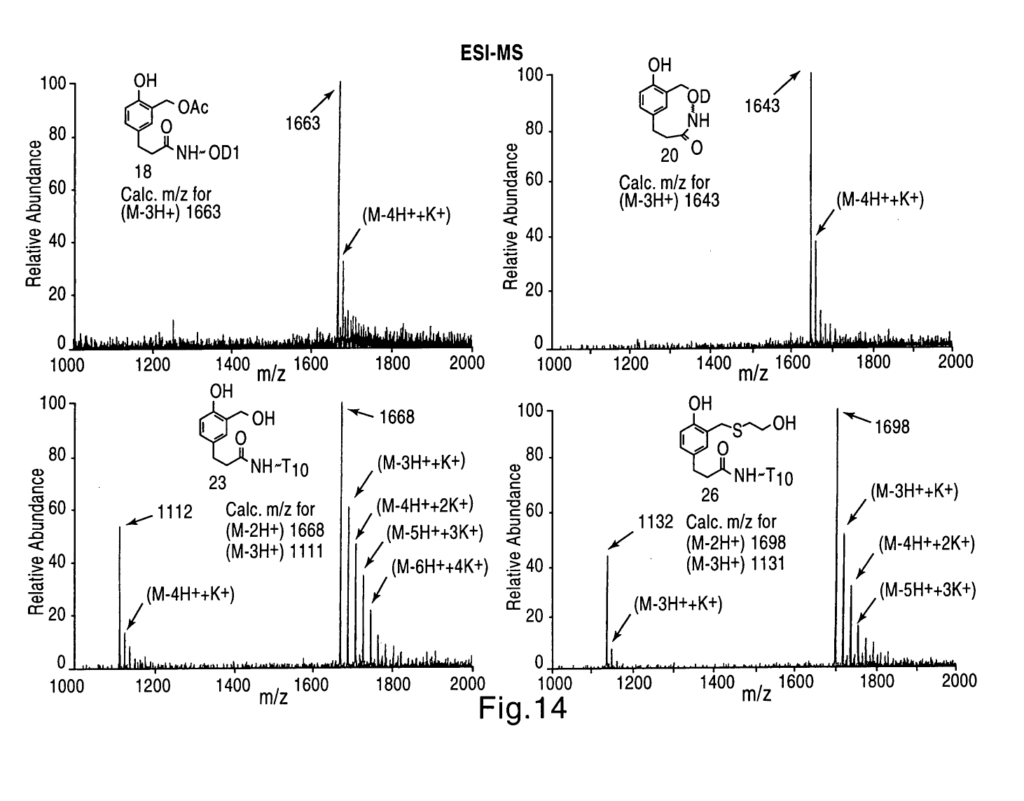

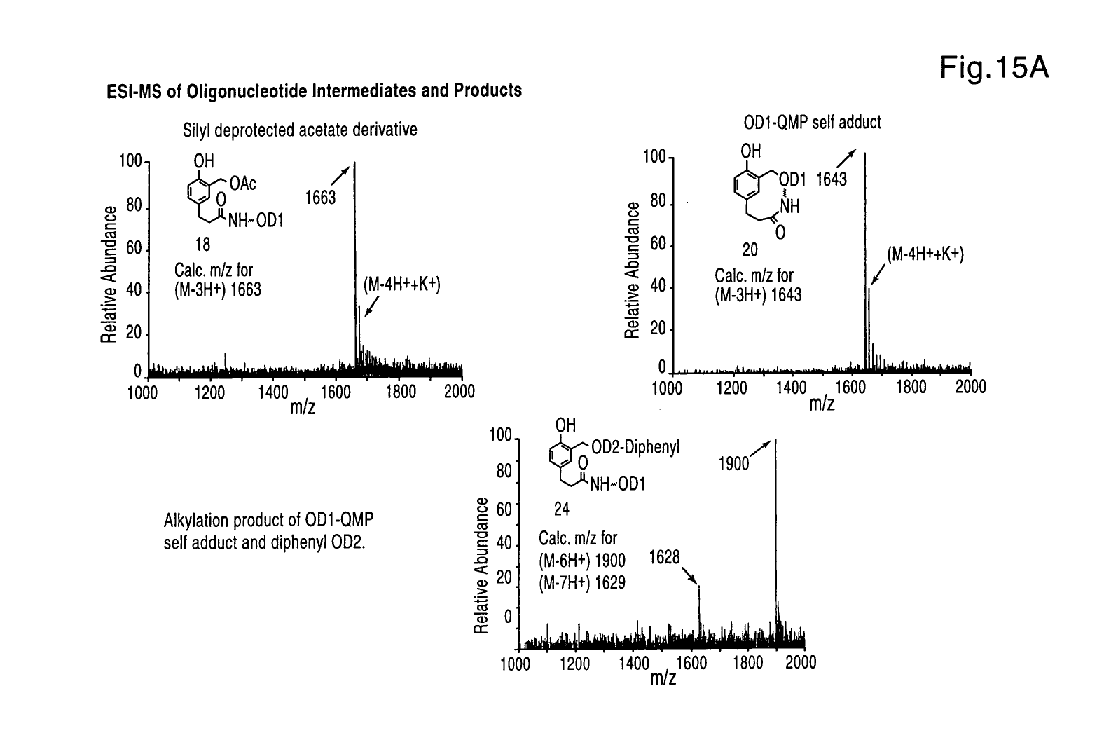

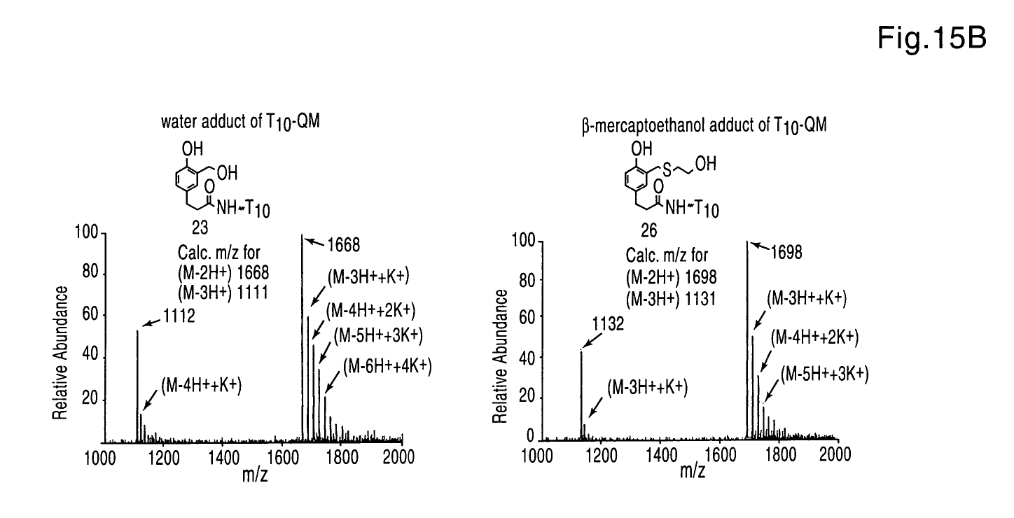

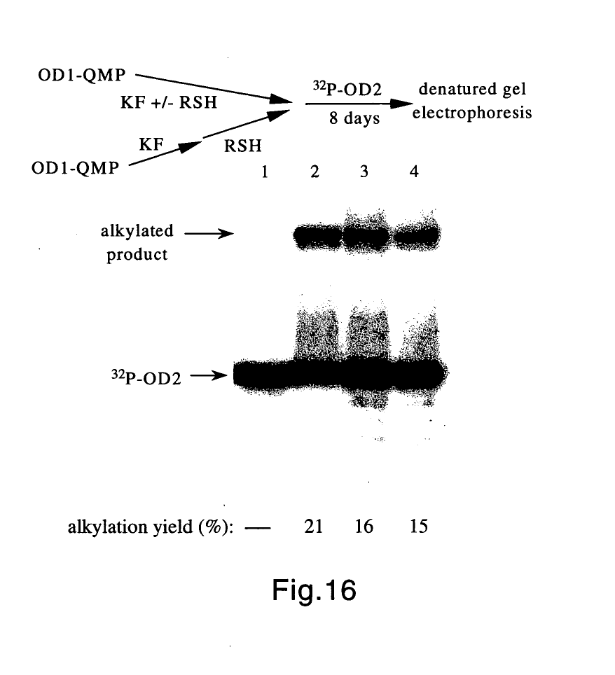

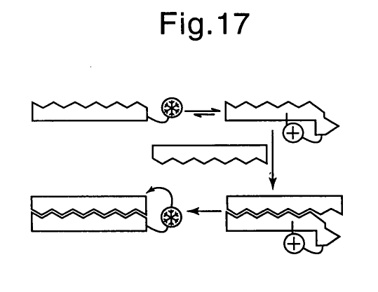

Thiols represent the strongest and most abundant nucleophiles in cells and yet even the model thiol was not competitive with intrastrand and intraduplex reaction of the nascent QM. This result is made all the more astonishing after considering that the rate constant for addition of_{.}-mercaptoethanol to the simple ortho QM under neutral conditions is more than 4-orders of magnitude greater than that of water, Modica et al., J. Org. Chem. 2001, 66, 41-52, and the closest nucleophile within the oligonucleotide conjugate (dA N1) is 26 atoms from the reactive exo-methylene position of the attached QM. See FIG. 11B.

Consistent with the electrophoretic analysis above, no adducts containing_{.}-mercaptoethanol were detected by HPLC analysis after incubation of this thiol, diphenyl-OD2 and the self adduct of OD1-QMP. Pre-incubation of this self-adduct with 1000 equivalents of the thiol for 6 days prior to adding diphenyl-OD2 reduced the yield of its alkylation after a final 8 day incubation from only 21% to 15%. In order to demonstrate the chemical competence of a thiol to react with a nascent oligonucleotide QM conjugate, the non-nucleophilic thymidine (T₁₀-QMP) was again used since it does not support formation of an intrastrand self-adduct. Treatment of this QM precursor with fluoride and 450 equivalents of_{.}-mercaptoethanol under aqueous conditions did indeed yield the thiol adduct (82%) and the water adduct (18%) as detected by A₂₆₀ and ESI-MS.

Intramolecular trapping of the QM intermediate is consequently more efficient for an attached oligonucleotide of mixed sequence (OD1) than intermolecular trapping by a strong thiol nucleophile. This is best explained as a kinetic phenomenon since once the thiol adduct formed, it remained stable as suggested by the constant ratio of water versus thiol addition during consumption of T₁₀-QMP. The ability of a thiol adduct to serve as a QMP likely depends on the stability of the nascent QM. In this example, the QM is too unstable to allow thiol expulsion, but a quinone methide derived from anthraquione had previously been shown to support reversible thiol addition. Angle et al., Tetrahedron Lett. 1992, 33, 6089-6092.

### Example 6

Target-activated alkylation of DNA. Alkylation of a chosen sequence of DNA by a self-adduct formed by a QM conjugate introduces a new approach to site-specific reactions. It has been shown that the conjugate may express minimal reaction over 8 days with competing nucleophiles and yet maintains an ability to alkylate a complementary strand. It is believed that this is accomplished by shifting the equilibrium between QM addition and elimination from an intrastrand to interstrand process by the thermodynamics of target recognition.

### Example 7

Oligonucleotide Conjugates. Oligonucleotide conjugates were formed by combining the appropriate activated succimidyl ester (1 mg) in CH₃CN/DMF (2:1, 400 μL) with the appropriate 5′-aminohexyloligonucleotide (20 nmol) in MOPS buffer (250 mM, pH 7.5, 400 μL) and incubating the mixture for 24 h at ambient temperature. The desired conjugates were purified by HPLC, dialyzed against water and lyophilized. ESI-MS (m/z) calcd for OD1-QMP (M-3H⁺)³⁻ 1701. Found 1701; calcd for (M-4H⁺)⁴⁺ 1276. Found 1276. ESI-MS (m/z) calcd for T₁₀-QMP(M-2H⁺)²⁻ 1746. Found 1746. ESI-MS (m/z) calcd for OD2-diphenylacetate (M-4H⁺)⁴⁻ 1618. Found 1618.

### Example 8

Self-adduct of the oligonucleotide conjugate (OD1-QMP). Reaction of OD1-QMP (2.2 μM) was initiated by desilylation with KF (1.0 M) in MES (20 mM, pH 7.0, 1.0 mL) and maintained at 25° C. Formation of the self-adduct was monitored by reverse phase (C-18) HPLC analysis using m-cresol (1.0 μM) as an internal standard. Conversion of OD1-QMP to its self-adduct was complete within 24 hr, purified by HPLC and confirmed by ESI-MS.

### Example 9

Nucleophilic quenching of the QM generated by T₁₀-QMP. Reaction of T₁₀-QMP (2.5 μM) was initiated by desilylation with KF (1.0 M) in MES (20 mM, pH 7.0) and maintained at 25° C. in the alternative presence and absence of_{.}-mercaptoethanol (1.1 mM). Reaction progress was monitored by reverse phase (C-18) HPLC analysis using m-cresol (1.0 μM) as an internal standard. Product formation was complete within 24 hr. In the absence of the thiol, only the water adduct of the QM intermediate was observed and confirmation by ESI-MS (m/z calcd for (M-2H⁺)²⁻ 1668. Found 1668; calcd for (M-3H⁺)³⁻ 1111. Found 1112.). In the presence of the thiol, both the thiol adduct (82%) and water (18%) adduct of the QM intermediate were observed (ESI-MS for the thiol adduct (m/z): calcd for (M-2H⁺)²⁻ 1698. Found 1698; calcd for (M-3H⁺)³⁻ 1131. Found 1132.). Yields were based on the integrated absorption at 260 nm measured during chromatographic analysis using the conditions described above.

### Example 10

Alkylation of oligonucleotide targets by OD1-QMP self-adduct. The self-adduct was generated in situ by incubating OD1-QMP (5.0 μM) with KF (1.0 M) in MES (20 mM, pH 7.0, 1.0 mL) for 2-8 days at 25° C. The desired conversion was confirmed by HPLC analysis. Target alkylation was then initiated by adding diphenyl-OD2 (2.5 μM) to the self-adduct (2.5 μM) in 20 mM MES pH 7 (25° C.) and reaction progress was monitored by HPLC analysis. Alternatively for electrophoretic analysis, the OD1-QMP self-adduct (2.2 μM) was generated equivalently. Reaction was initiated by addition of the self-adduct (1.1 μM) to 5′-[³²P]-OD2 (90 nCi, 1.0 μM) and maintained at ambient temperature for 8 days. Products were separated by denaturing gel electrophoresis (20% acrylamide, 7 M urea) and quantified by phospholmagery using ImageQuant software.

### Example 11

Formation of QM from its silyl precursor involves the initial removal of the t-butyldimethylsilyl (TBDMS) group and subsequent elimination of the benzylic leaving groups. See Scheme 1.

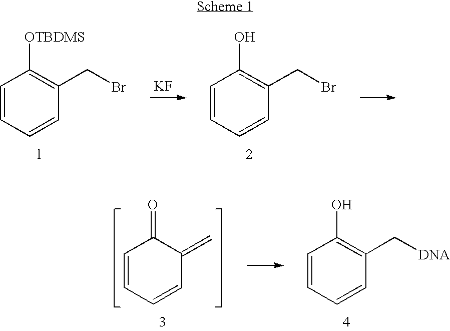

No DNA alkylation with a TBDMS-QM conjugate precursor was observed in the absence of fluoride ion, Zhou et al., Bioorg. Med. Chem. 2001, 9, 2347-2354, or if the TBDMS group of the precursor was replaced with a stable methyl ether, Zeng et al., J. Org. Chem. 1996, 61, 9080-9081, which indicates desilylation is a prerequisite for QM formation. Thus desilylation occurs in the reported DNA-induced alkylation with a hybridized TBDMS-QM precursor under physiological pH in the absence of fluoride ion. Li et al., J. Am. Chem. Soc. 1991, 113, 7771-7773; Li et al., Bioconjugate Chem. 1994, 5, 497-500. This implies that desilylation might be hybridization promoted since the general condition for the removal of TBDMS groups requires fluoride ion or acidic environments (pH<4), Davies et al., J. Chem. Soc., Perkin Trans. 1 1992, 3043-3048, which was subsequently investigated with a model derivative independent of QM formation and DNA alkylation.

The design of model derivatives is based on the assumption that if DNA hybridization promotes desilylation of 5, TBDMS group of 7 will be also removed upon hybridization. See Scheme 2.

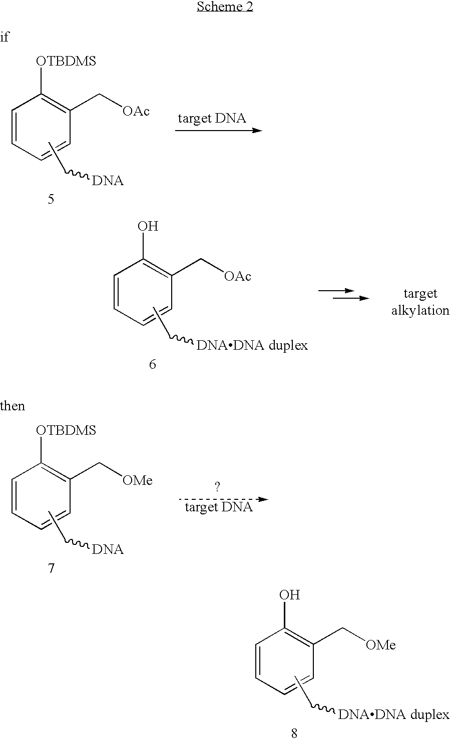

In Scheme 2, the acetate leaving group of precursor 5 was replaced with methoxyl groups, which are poor leaving groups and thus prevent the formation of quinone methide. A bismethoxy-substituted derivative 14 as the model was synthesized from bis(hydroxymethyl)phenylpropionic acid 9 due to synthetic convenience. See Scheme 3.

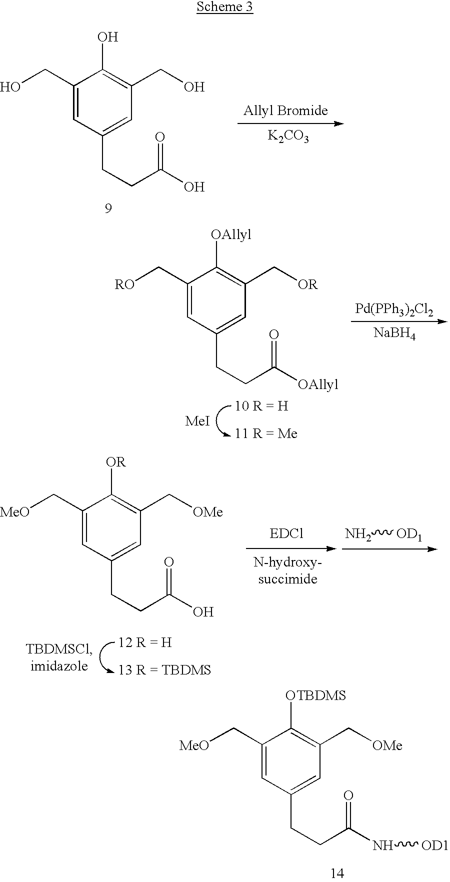

In Scheme 3, the phenol and acid moieties of 9 were selectively protected as their allyl ether and ester respectively, Martin et al., J. Org. Chem. 1982, 47, 1513-1518, allowing for methylation of hydroxymethyl groups by methyl iodide. Greene et al., J. Am. Chem. Soc. 1980, 102, 7583-7584. The allyl groups were then removed with catalytic Pd(PPh₃)₂Cl₂ in the presence of NaBH₄, Beugelmans et al., Tetrahedron Lett. 1994, 35, 4349-4350, and the resulting phenol was converted to its TBDMS ether 13. Corey et al., J. Am. Chem. Soc. 1972, 94, 6190-6191. Activation of 13 and subsequent coupling with an 5′-aminohexyl-oligonucleotide (H₂N-OD1) resulted in the desired TBDMS-methoxyconjugate 14 in 70% yield, as seen in Schemes 3 and 4. Zhou et al., Bioorg. Med. Chem. 2001, 9, 2347-2354.

The potential of hybridization promoted desilylation was assessed by comparing the desilylation rates of derivative 14 without KF in the presence or absence of a complementary strand OD2 at 25° C. See Scheme 4.

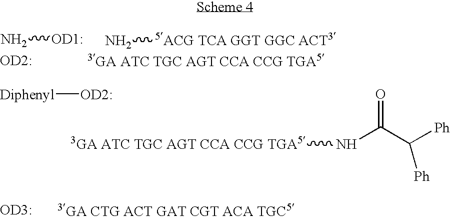

Desilylation was monitored by HPLC analysis following the consumption of 14 and the concurrent formation of desilylated product. See Schemes 3 and 4. No loss of 14 was observed in the presence or absence of OD2 after 4 days, which indicated that TBDMS ether of a DNA conjugate is highly stable under physiological conditions. As a comparison, the rates of KIF induced desilylation in the presence or absence of OD2 were also determined. The half-life of conjugate 14 with KF was about 90 mm in the absence of OD2 while the half-life of conjugate 14 was 140 mm with complementary OD2. The slower desilylation in the duplex DNA might be due to increased electrostatic repulsions between the phosphate backbone and F-. Hansler et al., J. Am. Cheni. Soc. 1993, 115, 8554-8557. These results indicated that desilylation of TBDMS group is not likely induced by hybridization of a TBDMS-conjugate with its complementary strand to generate QM for target DNA alkylation, which is inconsistent with previously interpretation. Li et al., J. Am. Chem. Soc. 1991, 113, 7771-7773; Li et al., Bioconjugate Chem. 1994, 5, 497-500.

Our rationale for target-induced alkylation, Li et al., J. Am. Chem. Soc. 1991, 113, 7771-7773; Li et al., Bioconjugate Chem. 1994, 5, 497-500, based on above results was that desilylation might occur during the isolation of QM-precursors prior alkylation study and target induced alkylation was attributed to reversible QM adducts formed after desilylation. To support this proposal, the homogeneity of QM precursor 5 was firstly examined through purification and isolation by HPLC analysis. No desilylation was observed during the coupling of activated ester with NH₂-OD1 and subsequent HPLC isolation. However, 67% of the precursor 5 was desilylated after lyophilizing the collected HPLC eluate, which agreed with the proposed origin of desilylation. Desilylation in lyophilization was probably due to an acidic environment contributed by ammonium acetate used as a HIPLC buffer. Hence, a dialysis step was introduced to removed the salt before lyophilization resulting in the final QM precursor 5 with a high homogeneity (>99%). Secondly, QM adducts were initially not considered to contribute target alkylation since QM hydrolysis products and nucleobase adducts were stable products. However, QM adducts have recently been shown to be reversible and serve themselves as QM precursors. See Scheme 5.

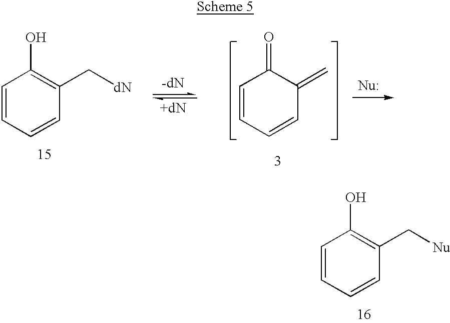

Angle and coworker showed that nucleosides were alkylated by a reversible QM from an anthrancene thiol-adduct. Angle et al., Tetrahedron Left. 1992, 33, 6089-6092. Freccero and coworkers reported that a reversible QM from a QM-lysine adduct was trapped by cysteine. Freccero et al., J. Org. Chem. 2001, 66, 41-52. It has now been also demonstrated that a kinetic QM N1-dA adduct converts to the thermodynamic N⁶-dA adducts through a reversible QM. Veldhuyzen et al., J. Am. Chem. Soc. 2001, 123, 11126-11132. Therefore, DNA induced alkylation can be achieved with QM regenerated from reversible QM nucleobase adduct upon hybridization, which was investigated and confirmed with the following model studies.

The formation of reversible QM nucleobase adduct after desilylation was investigated with a mono-alkylating QM derivative since it minimizes the range of possible products and competitive pathways in the reaction. A TBDMS-monoacetate-OD1 17 was synthesized using modified procedures as reported. Zhou et al., Bioorg. Med. Chem. 2001, 9, 2347-2354. Desilylation was initiated by adding KF to 17 in the absence of the complementary DNA and the progress of the reaction was monitored by HPLC and ESI-MS analysis. See FIG. 2. An initial product was observed after 30 mm and identified by ESI-MS analysis as the acetate derivative 18 with an observed mass (M-3H+)/3 signal of 1663 (calc. as 1663). Concurrently, derivative 17 was almost consumed. Derivative 18 was then converted to a broad signal as product 20 in >90% yield from 17 over 24 h at 25° C. and no other products were observed. See FIG. 2.

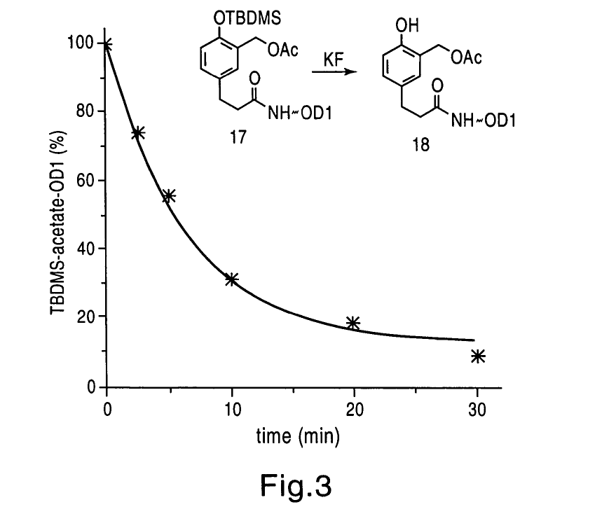

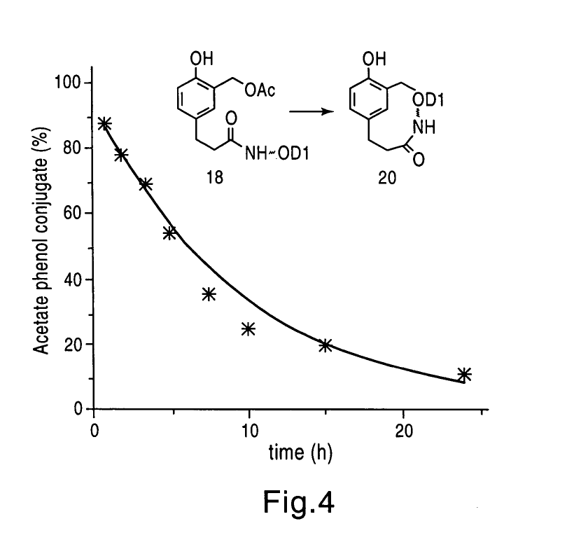

Subsequent ESI-MS analysis of the collected HIPLC eluate of product 20 showed a mass signal of 1643, which exactly matches the calculated (M˜3H+)/3 mass of QM intra-strand DNA nucleobase adduct. Desilylation of derivative 17 was observed as a pseudo first-order reaction with a rate of 0.16A0.01 min¹ (R²=0.993) and the consumption of 18 occurred about 100 times slower at rate of (1.8A0.3)×10³ min' (R²=0.963) as a pseudo first-order reaction. See FIGS. 3 and 4. QM-H₂O adduct, which can be formed through competitive hydrolysis pathway, has a calculated (M-3H˜)/3 mass of 1649 and was not detected by ESI-MS analysis. This indicated that product 20 appeared as intra-strand QM-DNA adduct not QM-H₂O adduct since these different (M-3H+/3 species can be well resolved by ESI-MS spectrometer.

Further confirmation of the preferred intra-strand reaction of QM over QM hydrolysis was to verify that QM-H20 adduct can be detected by both HPLC and ESI-MS analysis under similar conditions if hydrolysis of QM occurs. This was accomplished by fully eliminating nucleobase addition with a polythymine derivative since thymines are weaker nucleophiles than water. Pande et al., J. Am. Chem. Soc. 1999, 121, 6773-6779. With reference to Scheme 6, it is noted that TBDMS-monoacetate-T₁₀ 21 was synthesized similarly to that of 17.

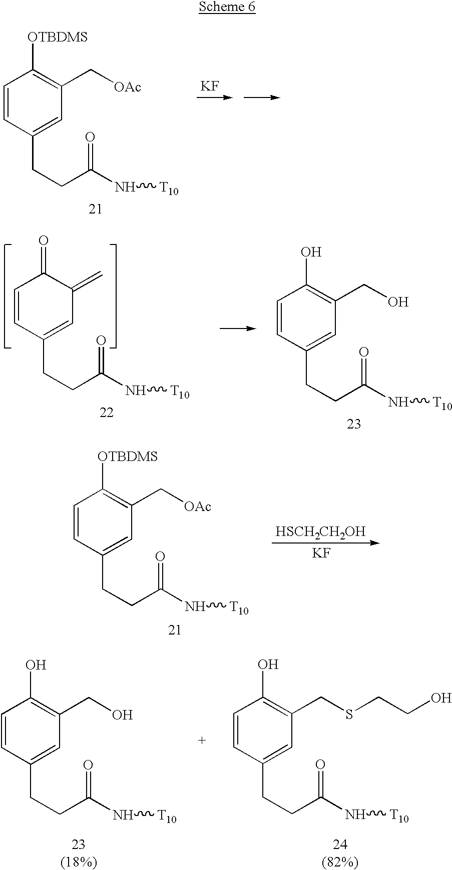

In Scheme 6, desilylation of 21 was initiated with KF under similar desilylation conditions of 17 and the progress of the reaction was monitored by HPLC and ESI-MS analysis. The expected QM-H₂O adduct 23 was formed in 98% yield from 21 after 24 h at 25° C. and confirmed with ESI-MS analysis by its observed (M-2H+)/2 mass of 1668 (calc. as 1668) and (M-3H+)/3 mass of 1112 (calc. as 1111). The observation of these mass signals indicated that QM-H₂O adduct can be directly detected by ESI-MS analysis if hydrolysis of QM occurred and also the observed mass signal for product 20 was for QM-DNA self adduct not as a result of dehydration of QM-H₂O adduct during MS analysis. These results concluded that intra-strand nucleobase addition on QM predominates the H₂O addition.

The formation of DNA self-adduct 20 was also examined in the presence of thiol to investigate any potential competition. This was accomplished by incubating derivative 17 with mercaptoethanol (450 equiv) in the presence of KF and the progress of the reaction was monitored by HPLC and ESI-MS analysis. QM-DNA self-adduct 20 was observed after 24 h at 25° C. and confirmed by ESI-MS analysis while no thiol adduct of 19 was detected. Further incubation of the reaction solution at 25° C. over 7 days found no loss of QM-DNA self-adduct 20 and the identity of 20 was also confirmed by ESI-MS analysis. To verify the detectablity of QM thiol adduct, TBDMS-acetate-T₁₀ 21 was treated similarly since DNA self-adduct formation is fully eliminated. Two products were formed after 24 h and identified by ESI-MS analysis as QM-H₂O adduct 23 and QM-thiol adduct 24 in 18% yield and 82% yield from 21, respectively. See Scheme 6. These results confirmed that if QM-thiol adduct is formed, it can be detected, and thus indicated that intra-strand nucleobase addition on QM is favored process even in the presence of strong nucleophiles in excess.

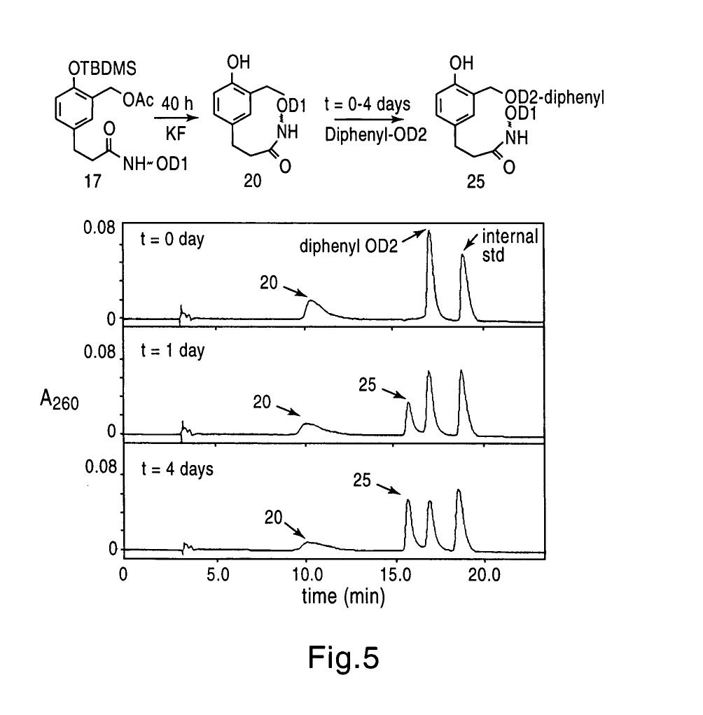

The reversibility of the resulting QM-DNA self-adduct 20 to regenerate QM upon hybridization was assessed by its ability to alkylate a target DNA. QM-DNA self-adduct 20 was formed by treating 17 (5.0 M) with KF in MES (pH 7.0, 20 mM) at 25° C. over 40 h and confirmed by HPLC analysis. Initially, alkylation of the complementary OD2 (Scheme 4) with self-adduct 20 was investigated. However, the retention time of OD2 on HPLC chromatograms (tr=11 min) overlapped with that of self-adduct 20. OD2 was then converted to its diphenylacetamide derivative, diphenyl-OD2 (Scheme 4), with a retention time of 17 mm, which did not overlap with any of other HPLC signals. Alkylation was initiated by adding QM-DNA self-adduct 20 (1.0 equiv) to target diphenyl-OD2 at 25° C. and the progress of the reaction was monitored by HPLC analysis. A new signal was observed by HPLC analysis over 4 days at 25° C. and was identified by ESI-MS analysis as the alkylated diphenyl-OD2 product 26 with observed mass of 1900 for (M-6H+)/6 (calc. as 1900) and of 1628 for (M-7H+)/7 (calc. as 1629). See FIG. 5. Alkylated diphenyl-OD2 product 25 was formed in 40% yield estimated from the mole extinction coefficients (260 nm) of 17, diphenyl-OD2 and 25 at day 4. Both diphenyl-OD2 and QM self-adduct 20 decreased concurrently to 60%. This target alkylation with DNA self-adduct 20 evidently demonstrated that QM is regenerated from reversible QM-DNA self-adduct 20 upon hybridization and the resulting QM is transferred to alkylate the target DNA. Alkylation through QM-DNA self-adduct might also explain the reported target-induced alkylation in the absence of KF, Li et al., J. Am. Chem. Soc. 1991, 113, 7771-7773; Li et al., Bioconjugate Chem. 1994, 5, 497-500, in which the self-adduct was formed through desilylation during the lyophilization process prior alkylation studies.

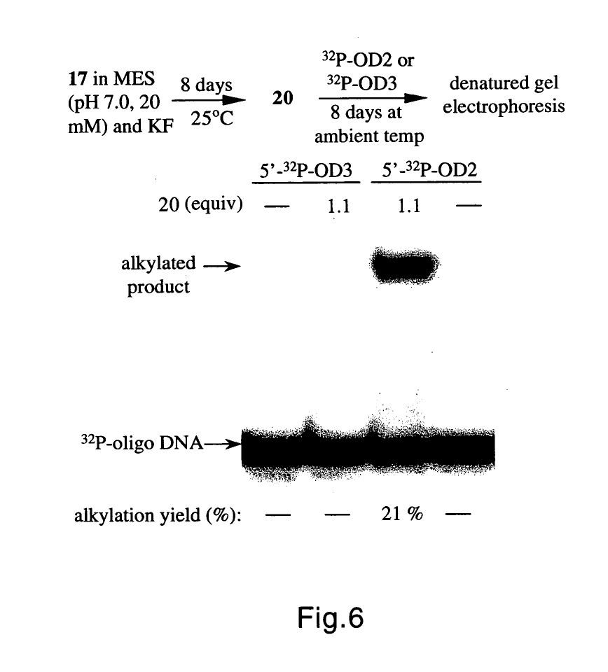

The selectivity of target DNA alkylation with self-adduct 20 was investigated by comparing alkylation of complementary OD2 with that of a non-complementary oligonucleotide with the same length (OD3). See Scheme 4. QM-DNA self-adduct 20 was prepared by treating 17 (2.2 M) in MES (pH 7.0, 20 mM) with KF and allowed to be incubated at 25° C. for 8 days so that the persistent capability of 20 to regenerate QM could be examined. The selectivity and efficiency of alkylation was tested by reacting self-adduct 20 (1.1 equiv) with ³²P-radiolabeled target OD2 or OD3 for 8 days at ambient temperature and quantified by gel analysis. No alkylation was observed with the non-complementary target OD3 while 22% of OD2 was alkylated by DNA self-adduct 20. See FIG. 6. This exclusive alkylation of OD2 versus OD3 suggested that only complementary DNA is alkylated by self-adduct 20 and thus the regeneration of QM from self-adduct 20 is a hybridization-induced process. In addition, the alkylating ability of self-adduct 20 is retained over 8 days.

Selective alkylation of OD2 by self-adduct 20 was next tested in the presence of non-complementary DNA competitors including OD3 and calf thymus DNA. Experiments were carried out similarly as described above and OD3 (10 equiv) or calf thymus DNA (10 equiv basepair of 20) was added along with ³²P-radiolabeled OD2. The reactions was incubated for 8 days at ambient temperature and gel analysis showed that DNA competitors did not decrease alkylation of the complementary target OD2. See FIG. 7. These results further substantiated that regeneration of QM from 20 through hybridization is favored over other intermolecular interactions.

Alkylation of target OD2 with self-adduct 20 was also assessed in the presence of a strong nucleophilic competitor such as mercaptoethanol, which may quench QM regenerated and thus decrease target alkylation. Alkylation studies was repeated as described above and mercaptoethanol (45 and 450 equiv relative to OD2) was added in the alkylation reaction. See FIG. 8. No decrease of the percentage alkylation of OD2 was found even in the presence of 450 equiv mercaptoethanol by gel analysis. Target alkylation with self-adduct 20 in the presence of mercaptoethanol was also confirmed by HPLC and ESI-MS analysis using diphenyl-OD2 as the target DNA under similar conditions while no QM thiol adduct was detected. These results suggested that QM regenerated through hybridization is preferably transferred to the complementary strand as an intramolecular process.

Since no effect of thiol was found in the above separated studies of self-adduct 20 formation and target alkylation by self-adduct 20, the presence of thiols should have no effect when these two processes are combined. This was verified by adding mercaptoethanol (450 equiv) to 17 prior to desilylation and subsequent self-adduct 20 formation and target alkylation were repeated as described above. Gel analysis showed no significant difference in target alkylation in the absence or presence of mercaptoethanol. The effect of thiol on the formed self-adduct 20 was also assessed by adding mercaptoethanol (450 equiv) at 48 h after desilylation was initiated. Subsequent alkylation was carried out similarly and no effect of thiol was observed on the target alkylation by self-adduct 20. These results demonstrate the high stability of self-adduct 20 in the absence of complementary targets and high selectivity and reactivity toward the complementary targets.

To determined how well alkylation by QM through self-adduct 20 is, alkylation by QM generated directly from QM precursor 17 was assessed as a comparison. See Scheme 6. This was accomplished by adding KF to a duplex DNA of derivative 17 and diphenyl-OD2 and the progress of the reaction was monitored by HPLC analysis. Acetate 18, alkylated diphenyl-OD2 product 26 and self-adduct 20 were all observed by HPLC analysis during the reaction course and their identities were confirmed by ESI-MS analysis. The desilylation rate of 17 in the presence of complementary DNA was determined to be (8.3A1.3)×10⁻² min⁻¹ (R²=0.984) as a pseudo first-order reaction and about a half of that in the absence of complementary DNA (0.16 mm⁻¹), which agreed with earlier observed slower desilylation in duplex. The consumption rate of 18 in duplex was also determined as (1.5A0.3)×10⁻³ min⁻¹ (R²=0.963) similar to that of 18 in the absence of complementary DNA (1.8×10⁻³ min⁻¹), which implied that formation of QM was not promoted or inhibited upon hybridization. The alkylated diphenyl-OD2 product 26 at day 4 was observed in 40% yield estimated from diphenyl-OD2, which is similar to that by self-adduct 20 as described above. This result suggested that target alkylation by QM regenerated is as efficient as direct alkylation.

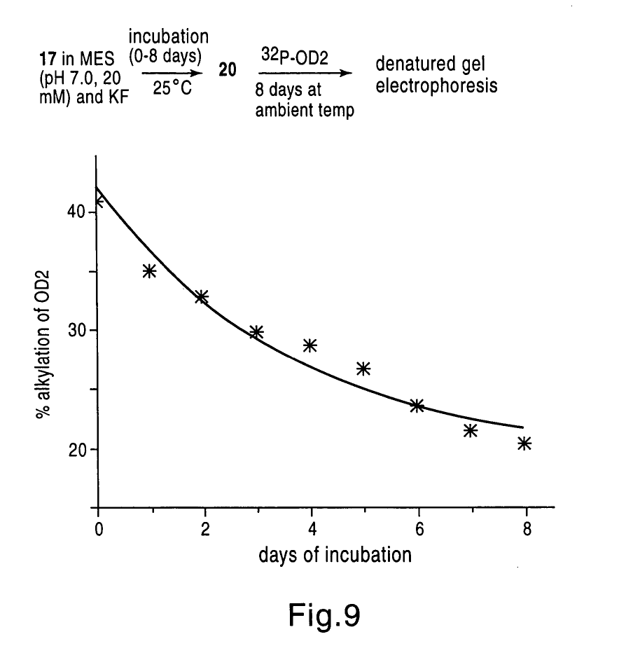

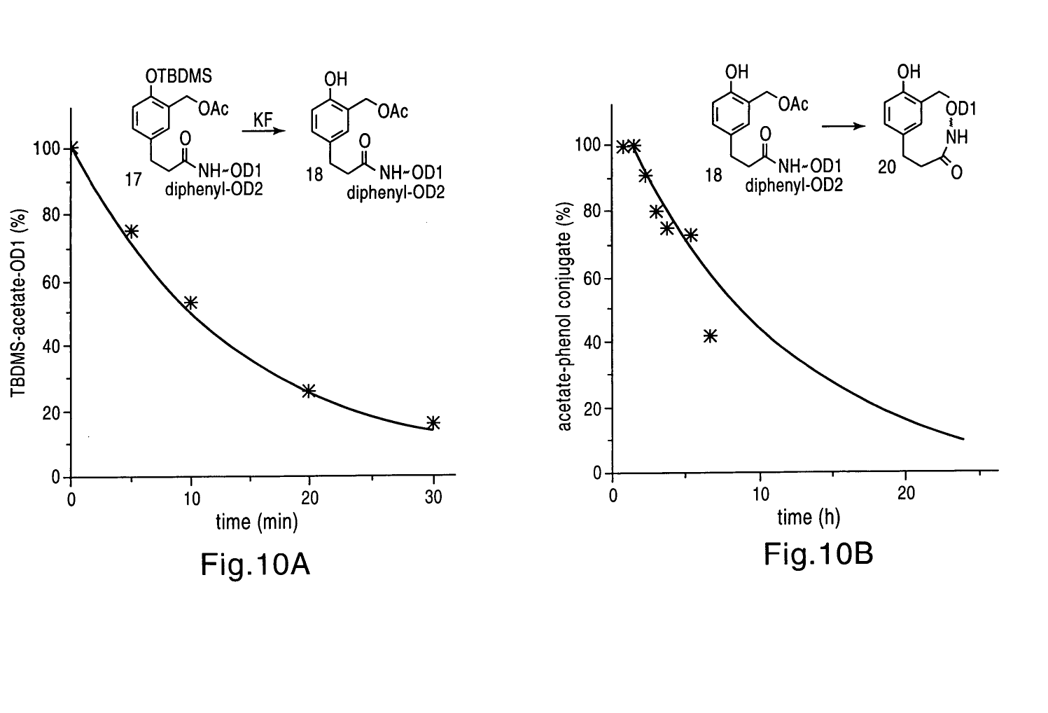

Although target alkylation was achieved in 40% yield by QM from self-adduct 20, there was QM-DNA self-adduct 20 remaining in the reaction and did not converted to product 26 upon further incubation. The identity of unreacted self-adduct 20 was confirmed by ESI-MS analysis, which suggested the heterogeneity for QM-DNA self-adduct 20 and may explain the broadness of HPLC signal of 20. Therefore, there is a reversible QM-DNA self-adduct which can regenerate QM upon hybridization and an irreversible QM-DNA self-adduct as a stable product. The existence of possible conversion of reversible self-adduct to irreversible one was investigated indirectly by comparing the remaining ability of QM-DNA self-adducts to alkylate a complementary target. This was achieved by incubating the formed self-adduct 20 in the absence of complementary DNA for various time (1-8 days) and subsequently analyzed with ³²P-radiolabeled OD2. See FIG. 6. Target alkylation of OD2 decreased from 38% to 22% when the incubation time increased from 1 to 8 days, which indicated that reversible self-adduct was converted to irreversible adduct over time. See FIG. 9. Interestingly, target alkylation of OD2 remained almost unchanged as 22% during the last 3 days of incubation. The loss of the alkylating ability of 20 followed a first order degradation with a rate of (2.1A0.4)×10⁻⁴ min¹⁻¹ (R²=0.959), and most significantly, there will be at least 20% target alkylation of OD2 if the incubation time was extrapolated to infinity. Since irreversible self-adduct can not regenerate QM and excess amount of mercaptoethanol has no effect on the target alkylation by self-adduct 20, the remaining 20% alkylating ability of 20 is not due to equilibrium interconversion among QM-DNA self-adducts. Therefore, there is a third latent self-adduct which is not quenched by other nucleophiles yet able to transfer QM to a complementary target selectively. A proposed mechanism for interconversion and subsequent alkylation of QM-DNA self-adduct 20 is illustrated in Scheme 8.

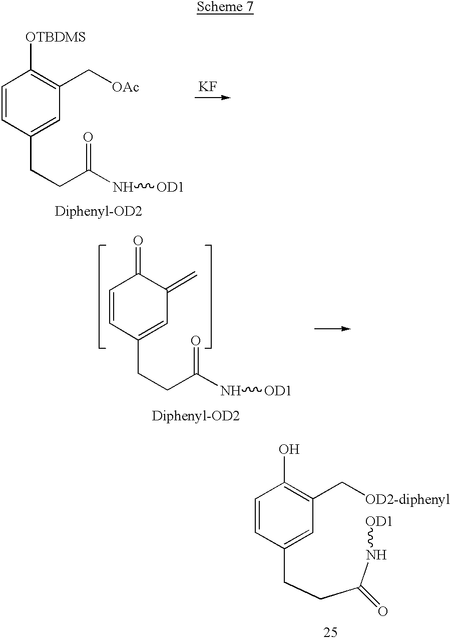

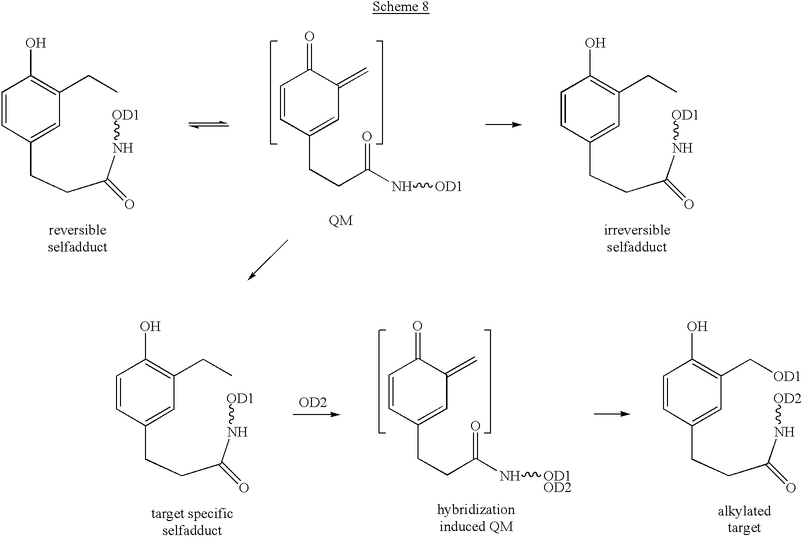

Reversible, irreversible and latent self-adducts may form from QM derivative in the absence of complementary target. Reversible self-adduct can regenerate QM while irreversible self-adduct remains as a stable adduct. Latent self-adduct is a stable product yet only regenerated QM upon hybridization and thus selectively alkylate the complementary target.

### Example 12

Allyl 3-(3′,S′-bis(hydroxymethyl)-4′-allyloxylphenyl)propionate (10). Allyl bromide (0.80 mL, 9.5 mmol) was added to a suspension of 9 (800 mg, 3.54 mmol) and K₂CO₃ (3.0 g) in dry DMF (10.0 mL) and the resulting mixture was stirred at room temperature under N₂ for 20 h. The reaction solution was then diluted with H₂O (150 mL) and extracted with diethyl ether (3×150 mL). The organic layers were collected, dried over MgSO₄ and concentrated. The resulting residue was purified by a silica gel flash column (0.5-3% MeOH in CH₂Cl₂) and yielded the desired 10 as a colorless oil (512 mg, 47%): ¹H NMR (CDC1₃, 400 MHz) 7.15 (s, 2H), 6.08 (m, 1H), 5.89 (m, 1H), 5.19-5.43 (m, 4H), 4.68 (s, 4H), 4.56 (d, J=5.6 Hz, 2H), 4.42 (d, J=5.6 Hz, 2H), 2.92 (t, J=7.6 Hz, 2H), 2.63 (t, J=7.6 Hz, 2H); ¹³C NMR (CDCl₃, 100.6 MHz) 172.5, 153.3, 136.8, 134.1, 133.5, 132.1, 128.6, 118.3, 117.9, 75.6, 65.2, 60.9, 35.7, 30.3; HRMS calcd. for C₁₇H₂₂O₅ (M+) 306.1467. Found 306.1469.

### Example 13

Allyl 3-(3˜,5′-bis(methoxymethyl)-4′-allyloxylphenyl)propionate (11). Excess iodomethane (6.0 mL) and Ag₂O (2.0 g) was added to a solution of 10 (350 mg, 1.14 mmol) in CH₃CN (2.0 mL) and the resulting suspension was refluxed under N₂ for 20 h. The reaction solution was filtered through a pad of MgSO₄ with ether and concentrated to yield the desired 11 as a colorless oil (362 mg, 95%): ¹H NMR (CDCl₃, 400 MHz) 7.17 (s, 2H), 6.06 (m, 1H), 5.87 (m, 1H), 5.18-5.42 (m, 411), 4.55 (d, J=5.6 Hz, 211), 4.68 (s, 4H), 4.43 (d, J=5.6 Hz, 2H), 3.39 (s, 611), 2.91 (t, J=8.0 Hz, 211), 2.65 (t, J=8.0 Hz, 2H). ¹³C NMR (CDCl₃, 100.6 MHz) 172.5, 154.0, 136.3, 133.9, 132.2, 131.4, 129.3, 118.1, 117.1, 76.0, 69.6, 65.1, 58.3, 35.8, 30.4; HRMS calcd. for C₁₉H₂₆O₅ (M+) 334.1780. Found 334.1771.

### Example 14

3-(3′,5′-Bis(methoxymethyl)-4′-hydroxyphenyl)propionic acid (12). Pd(PPh₃)₂Cl₂ (10 mg, 14 mol) was added to a solution of 11(600 mg, 1.80 mmol) in dry THF (10 nIL) and then NaBH₄ (200 mg) was added after the suspension was stirred for 5 mm under N₂. The resulting suspension was stirred under N₂ at room temperature for 20 h and quenched with saturated aqueous NH₄Cl (80 mL). The solution was adjusted to pH 11 with 1 N NaOH and washed with ether (2×100 mL). The aqueous layer was collected, acidified with 1 N HC1 and extracted with diethyl ether (3×150 mL). The organic layers were collected, dried over MgSO₄ and concentrated and yielded the desired product 12 as a colorless oil (282 mg, 62%): ¹H NMR (CDCl₃, 400 M7 Hz) 6.94 (s, 211), 4.54 (s, 411), 3.41 (s, 611), 2.89 (t, J=7.6 Hz, 2H), 2.60 (t, J=7.6 Hz, 211), ¹³C NMR (CDCl₃, 100.6 MHz) 178.8, 152.6, 131.2, 128.3, 123.5, 71.8, 58.3, 35.9, 29.7; HRMS calcd. for C₁₃H₁₈O₅ (M+) 254.1154. Found 254.1157.

### Example 15

3-(3′,5′-Bis(methoxymethyl)-4′-t-butyidimethylsilyloxyphenyl)propionic acid (13).

N,N-Diisopropyl-N-ethylamine (500 _L, 2.88 mmol) was added to a solution of 12 (258 mg, 1.02 mmol) and t-butyldimethylsilyl chloride (1.00 g, 6.63 mmol) in dry DMF (5.0 mL) and the resulting solution was stirred under N₂ for 20 h. The reaction solution was then diluted with ether (150 mL), washed with 1120 (3×150 mL) and brine (2×150 mL). The organic layer was concentrated and the residue was dissolved in MeOH (10.0 mL). Potassium carbonate (300 mg) was added and the resulting suspension was stirred for 2 h. The reaction solution was diluted with 1120 (100 niL), adjusted to pH 5 with 0.1 N HCl and extracted with ether (2×100 mL). The organic layers were collected, dried over MgSO₄ and concentrated. The resulting residue was purified by a silica gel flash column (25-35% EtOAc in hexanes) and yielded the desired 13 as a colorless oil (192 mg, 51%): ¹H NMR (CDCl₃, 400 MHz) 7.14 (s, 211), 4.40 (s, 411), 3.35 (s, 611), 2.86 (t, J=8.4 Hz, 211), 2.64 (t, J=8.4 Hz, 211), 1.01 (s, 911), 0.15 (s, 611); ¹³C NMR; (CDC1₃, 100.6 MHz) 178.5, 148.8, 133.5, 129.0, 128.4, 69.6, 58.2, 35.7, 29.9, 26.0, 18.7, −3.8; HRMS calcd. for C₁₉H₃₂Q₅Si (M+) 368.2019. Found 368.2026.

### Example 16

TBDMS-methoxy-OD1 (14). A solution of 13 (168 mg, 0.46 mmol), 1-(3-dimethyl-aminopropyl)-3-ethylcarbodiimde hydrochloride (EDCl, 105 mg, 0.55 mmol) and N-hydroxysuccimide (64 mg, 0.55 mmol) in DMF (5.0 mL) was stirred under N₂ at ambient temperature for 20 h. The reaction solution was diluted with 1120 (100 mL) and extracted with ether (2×100 mL). The organic layers were collected, washed with brine (100 mL), dried over MgSO₄ and concentrated. The resulting residue was purified by a silica gel flash column (20-30% EtOAc in hexanes) and yielded the desired activated ester as a colorless oil (116 mg, 54%): ¹H NMR (CDCl₃, 400 MHz) 7.15 (s, 2H), 4.38 (s, 411), 3.35 (s, 6H), 2.96 (m, 211), 2.88 (m,211), 2.79 (s, 411), 1.00 (s, 911), 0.13 (s, 611); ¹³C NMR (CDCl₃, 100.6 MHz) 169.1, 168.0, 149.0, 132.5, 129.3, 128.4, 69.5, 58.3, 32.7, 29.7, 26.0, 25.6, 18.7, −3.8; HRMS calcd. for C₂₃H₃₆O₇NSi (M+) 446.2261. Found 446.2253.

A solution of excess activated ester (1 mg) in CH₃CN/DMF (2:1, 400 L) was added to a solution of 5-aminohexyloligonucleotide-OD1 (20 nmol) in 3-(N-morpholino)-propanesulfonate (MOPS, pH 7.5, 250 mM, 400 L) and left at ambient temperature for 24 h. The desired conjugated 14 was purified by HPLC in 75% yield from the initial oligonucleotide estimated by UV absorbance at 260 nm. The homogeneity of 14 was further confirmed after the collected HPLC eluate was dialyzed and lyophilized. ESI-MS (m/z) calcd for (M-3H+)³ 1706. Found 1706; calcd for (M-4H+)⁴ 1280. Found, 1280.

### Example 17

3-(3-t-Butyldimethylsilyoxymethyl-4-t-butyidimethylsilyoxyphenyl)propionic acid.

A solution of 37% formaldehyde (6.0 mL) was added to 3-(4-hydroxyphenyl)propionic acid (2.0 g, 12 mmol) and the pH of the solution was adjusted to pH 8 with 10% aqueous NaOH solution. The resulting solution was heated at 65° C. for 24 h and a precipitate was formed when acetone (1000 mL) was added. The solid was collected, dried under vacuum and dissolved in dry DMF (50 mL). t-Butyldimethylsilyl chloride (7.5 g, 50 mmol) and imidazole (3.5 g, Si mmol) were added and the resulting suspension was stirred under N₂ for 20 h. The reaction was worked up with ether (500 mL) and a diluted HCl solution (pH 3, 500 mL). The organic layer was collected and concentrated. The resulting residue was dissolved in MeOH (20 mL) and K₂CO₃ (2.0 g) was added. The suspension was stirred for 3 h and worked up with ether (200 mL) and diluted HCL solution (pH 4, 200 mL). The resulting residue was purified by a silica gel flash column (6-8% EtOAc in hexanes containing 0.5% HOAc) and yielded the desired acid as a colorless oil (636 mg, 13%). 111 NMR (400 MHz, CDCl₃) 7.26 (s, 111), 6.92 (d, 111, J=8 Hz) 6.63 (d, 111, J=8 Hz), 4.7 (s, 211), 2.88 (t, 211, J=7.6 Hz), 2.63 (t, 2H, J=7.6 Hz), 0.97 (s, 911), 0.93 (s, 911), 0.18 (s, 611), 0.08 (s, 611); ¹³C NMR (100.6 MHz) 177.6, 150.4, 132.6, 132.1, 126.9, 126.7, 117.8, 60.5, 35.6, 30.1, 26.0, 25.7, 185, 18.2, −4.3, −5.4; HRMS calcd. for C₂₂H₄₀O₄Si₂ (M+) 3424.2465. Found 424.2474.

### Example 18

Succimidyl 3-(3-acetyloxymethyl-4-t-butyidimethylsilyoxyphenyl)propionate. FeCl₃.6H₂O (15 mg, 0.055 mmol) was added to a solution of the above acid (200 mg, 0.48 mmol) in acetic anhydride (3.0 mL) at 0° C. and the resulting yellow solution was stirred at ambient temperature for 30 mm. The reaction was diluted with ether (100 mL) and washed with 1120 (100 mL) and saturated NaHCO₃solution (100 mL). The organic layer was collected, dried over MgSO₄ and concentrated. The resulting oil was dissolved in DMF (10 mL) and EDCI (100 mg, 0.52 mmol), N-hydroxysuccimide (65 mg, 0.57 mmol) was added. The reaction was stirred under N₂ for 20 h. The reaction solution was worked up with ether (100 mL) and 1120 and concentrated. The resulting residue was purified by a silica gel flash column (10-45% EtOAc in hexanes) and yielded the desired product as a colorless oil (51 mg, 24%). ¹H NMR (400 MHz, CDC1₃) 7.13 (s, 1H), 7.02 (d, 1H, J=8 Hz) 6.73 (d, 1H, J=8 Hz), 5.05 (s, 2H), 2.96 (t, 2H, J=7.6 Hz), 2.86 (t, 2H, J=7.6 Hz), 2.80 (s, 4H), 2.06 (s, 3H), 0.96 (s, 9H), 0.20 (s, 6H); ¹³C NMR 170.9, 169.1, 167.9, 152.7, 131.7, 130.1, 129.2, 126.6, 118.7, 62.1, 32.8, 29.6, 25.6, 25.6, 21.0, 18.2, −4.3; HRMS calcd. for C₂₂H₃₁O₇NSi (M+) 449.1870. Found 449.1856.

### Example 19

TBDMS-acetate-OD1 (17). A solution of excess succimidyl 3-(3-acetyloxymethyl-4-t-butyldimethylsilyoxyphenyl)propionate (1 mg) in CH₃CN/DMF (2:1, 400 L) was added to a solution of 5′-aminohexyloligonucleotide-ODI (20 nmol) in MOPS buffer (250 mM, pH 7.5, 400 L) and left at ambient temperature for 24 h. The desired conjugated 17 was purified by HPLC in 72% yield from the initial oligonucleotide estimated by UV absorbance at 260 nm. The homogeneity of 17 was further confirmed after the collected HPLC eluate was dialyzed and lyophilized. ESI-MS (m/z) calcd for (M-3H+)³ 1701. Found 1701; calcd for (M-4H+)⁴ 1276. Found, 1276.

### Example 20

TBDMS-acetate-T₁₀ (21). A solution of excess succimidyl 3-(3-acetyloxymethyl-4-t-butyidimethylsilyoxyphenyl)propionate (1 mg) in CH₃CN/DMF (2:1, 400 L) was added to a solution of 5′-aminohexyloligonucleotide-T₁₀ (20 nmol) in MOPS buffer (pH 7.5, 250 mM, 400 L) and left at ambient temperature for 24 h. The desired conjugated 21 was purified by HPLC in 40% yield from the initial oligonucleotide estimated by UV absorbance at 260 nm. The homogeneity of 21 was further confirmed after the collected HPLC eluate was dialyzed and lyophilized. ESI-MS (m/z) calcd for (M2H+)² 1746. Found, 1746; calcd for (M-3H+)³ 1164. Found, 1164.

### Example 21

Diphenyl-OD2. A solution of EDCI (345 mg, 1.80 mmol), N-hydroxysuccimide (210 mg, 1.80 mmol) and diphenylacetic acid (318 mg, 1.50 mmol) in DMF (5.0 mL) was stirred under N₂ for 20 h. The reaction solution was diluted with ether (100 mL) and washed with H20. The organic layer was collected and concentrated. The desired succimidyl ester was crystallized with EtOAc/hexanes as a white solid (223 mg, 48%) with mp 120-122° C. 111 NMR (400 MHz, CDCl₃) 7.32 (m, 1OH), 5.33 (s, 1H), 2.80 (s, 4H); ¹³C NMR (CDC1₃, 100.6 Mhz) 168.9, 168.1, 136.7, 128.8, 128.7, 127.9, 54.0, 25.6; HRMS calcd for C₁₈H₁₆NO₄ (M+H+) 310.1079. Found 310.1069.

A solution of excess succimidyl ester (1 mg) in CH₃CN/DMF (2:1, 400 L) was added to a solution of 5′-aminohexyloligonucleotide OD2 (20 nmol) in MOPS buffer (pH 7.5, 250 mM, 400 L) and left at ambient temperature for 48 h. The desired diphenyl-OD2 was purified by HPLC in 52% yield from the initial oligonucleotide estimated by UV absorbance at 260 nm. The homogeneity of desired diphenyl-OD2 was further confirmed after the collected HPLC eluate was dialyzed and lyophilized. ESI-MS (m/z) calcd for (M-4H+)⁴ 1618. Found 1618.

### Example 22

Desilylation of TBDMS-methoxy-OD1 (14) in the absence of KF for potential hybridization promotion. Desilylation was initiated by adding TBDMS-methoxy-OD1 14 (2.5 M) to KCl (200 mM) and morpholineethanesulfonate (MES, pH 7.0, 20 mM) in the absence or presence of OD2 (2.5 M). The progress of reaction at 25° C. was monitored by HPLC analysis using m-cresol (1.0 M) as an internal standard. As a controlled study of KF-induced desilylation of 14, reactions in which KCl was replaced by KF (200 mM) were also monitored under similar conditions.

### Example 23

Formation of DNA self-adduct from TBDMS-acetate-OD1 (17). Desilylation was initiated by adding KF (1.0 M) to TBDMS-acetate-OD1 17 (2.2 M) in MES (pH 7.0, 20 mM) in the absence of the complementary DNA and the progress of reaction at 25° C. was monitored by HPLC analysis using m-cresol (1.0 M) as an internal standard. The formation of acetate 18 and QM-DNA self-adduct 20 was confirmed by ESI-MS analysis of the collected HPLC eluate. The amount of 17 and 18 in the reaction solution over time was determined based on the area integration of corresponding HPLC signals to that of internal standard, which subsequently afforded the rates of consumption as calculated by Microcal Origin 6.0 software (Microcal Software, Inc., Northampton, Mass.). To assess the potential of competitive additions by other nucleophiles versus QM-DNA self-adduct formation, desilylation of 17 (2.5 M) was also initiated in the presence of KF (1.0 M) and mercaptoethanol (1.125 mM) under similar conditions. After 24 h, only QM-DNA self-adduct 20 was observed by HPLC analysis and the identity was confirmed ESI-MS analysis. QM-thiol adduct were not detected by either HPLC or ESI-MS analysis. QM-DNA self adduct 20 was also detected as the only QM adduct in the presence of mercaptoethanol by HPLC and ESI-MS analysis when the incubation time was increased to 7 days at 25° C.

### Example 24

Desilylation of TBDMS-acetate-T₁₀ (21) in the absence and presence of thiols. Desilylation was initiated by adding KF (1.0 M) to 21(2.5 M) in MES (pH 7.0, 20 mM) in the absence of the complementary DNA and the progress of reaction at 25° C. and was monitored by HPLC analysis using m-cresol (1.0 M) as an internal standard. A signal as QM hydrolysis product 23 was observed after 24 h and confirmed by ESIMS analysis while that of 21 was completely consumed. To assess the potential of competitive additions by other nucleophiles versus QM hydrolysis, desilylation of 21 (2.5 M) was also initiated in the presence of KF (1.0 M) and mercaptoethanol (1.125 mM) under similar conditions. A signal as QM-thiol adduct 24 was observed after 24 h by HPLC analysis in 82% yield from 21 based on the area integration to that of internal standard while the signal of hydrolysis product 23 was found in 18% yield and the identities of these signals were confirmed by ESI-MS analysis. Anal. Data for 26: ESI-MS (m/z) calcd for (M-2H˜)² 1698. Found 1698; calcd for(M-3H+)³-1131. Found 1132.

### Example 25

Alkylation of diphenyl-OD2 by self-adduct (20). QM-DNA self-adduct was formed by treating 17 (5.0 M) with KF (1.0 M) in MES (pH 7.0, 20 mM) for 40 h at 25° C. and confirmed by HIPLC analysis. Alkylation was initiated by adding diphenyl-OD2 (2.5 M) to the resulting 20 in MES (pH 7.0, 20 mM) and the progress of the reaction was monitored over a period of 4 days. The formation of alkylated diphenyl-OD2 product 25 was confirmed by ESI-MS analysis of the collected HPLC eluate.

### Example 26

Oligonucleotide radiolabeling and denatured gel electrophoresis analysis. Oligonucleotides were labeled at their 5′ terminus with 5′-_-³²P-ATP (Amersham Pharmacia, Piscataway, N.J.) and T4 polynucleotide kinase (New England Biolabs, Beverly, Mass.) as directed by supplier. Product analysis of radiolabeled target was achieved by denatured polyacrylamide gel electrophoresis (20%, 7M urea) and Phospholmage analysis (Molecular Dynamic, Sunnyvale, Calif.). The loading solution contained 50 nCi per lane with 0.05% xylene cyanole FF and 0.05% bromophenol blue in 80% formamide. All the experiments were at least duplicated.

### Example 27

Selective alkylation of OD2 versus OD3 by QM-DNA self-adduct (20) by gel electrophoresis analysis. QM-DNA self-adduct was formed by treating 17 (2.2 M) with KF (1.0 M) in IVIES (pH 7.0, 20 mM) for 8 days at 25° C. and confirmed by HPLC analysis. Alkylation was initiated by adding ³²P-radiolabeled OD2 (1.0 M) or ³²P-radiolabeled OD3 (1.0 M) to the resulting adduct 20 (1.1 M) in MES (pH 7.0, 20 mM) and left for 8 days at ambient temperature. The percent alkylation of ³²P-radiolabeled target was quantified by 20% denatured gel electrophoresis analysis.

### Example 28

Alkylation by QM-DNA self-adduct (20) in the presence of DNA competitors by gel electrophoresis analysis. QM-DNA self-adduct was formed by treating 17 (2.2 M) with KF (1.0 M) in MES (pH 7.0, 20 mM) for 8 days at 25° C. and confirmed by HPLC analysis. Alkylation was initiated by adding ³²P-radiolabeled OD2 (1.0 M) and non-radiolabeled DNA competitors including OD3 (10 M), duplex or denatured calf thymus DNA (ctDNA, 10 M) to the resulting adduct 20 (1.1 M) in MES (pH 7.0, 20 mM). Denatured ctDNA was obtained by cooling a hot solution of duplex ctDNA (90° C. for 2 mm) directly on ice. The reaction was left for 8 days at ambient temperature and the percent alkylation of ³²P-radiolabeled target was quantified by 20% denatured gel electrophoresis analysis.

### Example 29

Alkylation of target OD2 by QM-DNA self-adduct (20) in the presence of thiol. DNA self-adduct was formed by incubating 17 (2.2 M) with KF (1.0 M) in MIS (pH 7.0, 20 mM) for 8 days at 25° C. and confirmed by HPLC analysis. Alkylation was initiated by adding ³²P-radiolabeled OD2 (1.0 M) and mercaptoethanol (45 or 450 M) to the resulting adduct 20 (1.1 M) in MES (pH 7.0, 20 mM) and left at ambient temperature for 8 days. The percent alkylation of ³²P-radiolabeled target was quantified by 20% denatured gel electrophoresis analysis. This result was also confirmed by HIPLC analysis using QM-DNA self-adduct 20 (2.5 M), diphenyl-OD2 (2.5 M) and mercaptoethanol (1.125 mM) in MES (pH 7.0, 20 mM) and KF (1.0 M) over 8 days at 25° C.

### Example 30

QM-DNA self-adduct formation and subsequent target alkylation in the presence of thiol. Desilylation was initiated by adding KF (1.0 M) and mercaptoethanol (0.99 mM) to 17 (2.2 M) in MES (pH 7.0, 20 mM) and left for 8 days at 25° C. Alkylation was initiated by adding ³²P-radiolabeled OD2 (1.0 M) to the resulting adduct 20 (1.1 M) in MES (pH 7.0, 20 mM) and left at ambient temperature for 8 days. The percent alkylation of ³²P-radiolabeled target was quantified by 20% denatured gel electrophoresis analysis. As a comparison to assess the effect of thiol on adduct 20, mercaptoethanol (0.99 mM) was added to a reaction solution of 17 (2.2 M) in MES (pH 7.0, 20 mM) and KF (1.0 M) at 48 h at 25° C. The resulting reaction was left at 25° C. for 6 days. Alkylation was initiated by adding ³²P-radiolabeled OD2 (1.0 M) to the resulting adduct 20 (1.1 M) using condition as described above and quantified similarly by gel analysis.

### Example 31

Alkylation of diphenyl-OD2 by TBDMS-acetate conjugate (17) in the presence of KF. Alkylation was initiated by adding KF (1.0 M) to 17 (2.5 M) and diphenyl-OD2 (2.5 M) in MES (pH 7.0, 20 mM) at 25° C. and the progress of reaction was monitored by HPLC analysis. The amount of 17 and 18 formed in the reaction over time was determined based on the integration of corresponding HPLC signals to that of internal standard, which subsequently afforded the rates of consumption as calculated by Microcal Origin 6.0 software.

### Example 32

Interconversion of QM-DNA self-adducts by gel electrophoresis analysis. Desilylation was initiated by adding KF (1.0 M) to 17 (2.2 M) in MES (pH 7.0, 20 mM) for 0-8 days at 25° C. Alkylation was initiated by adding ³²P-radiolabeled OD2 (1.0 .M) to the resulting DNA solution (1.1 M) in MIS (pH 7.0, 20 mM) and the reaction was left at ambient temperature for 8 days. The percent alkylation of ³²P-radiolabeled target was quantified by 20% denatured gel electrophoresis analysis and subsequently analyzed by Microcal Origin 6.0 software.

### Example 33

Synthesis of N-hydroxysuccimidyl esters for conjugation to 5′-aminohexyloligonucleotides 3-(3-t-Butyldimethylsilyoxymethyl-4-t-butyldimethylsilyoxyphenyl)propionic acid. A solution of 37% formaldehyde (6.0 mL) was added to 3-(4-hydroxyphenyl)propionic acid (2.0 g, 12 mmol) and the pH of the solution was adjusted to pH 8 with 10% aqueous NaOH solution. The resulting solution was heated at 65° C. for 24 h. After cooling, acetone (1 L) was added to precipitate the crude product. The solid was collected, dried under vacuum and dissolved in dry DMF (50 mL). t-Butyldimethylsilyl chloride (7.5 g, 50 mmol) and imidazole (3.5 g, 51 mmol) were added and the resulting suspension was stirred under N₂ and ambient temperature for 20 h. The reaction mixture was diluted with ether (500 mL) and 1 mM HCl (500 mL). The organic layer was collected and concentrated. The resulting residue was dissolved in MeOH (20 mL) and K₂CO₃ (2.0 g) was added. The suspension was stirred for 3 h, quenched with 0.1 mM HCl (200 mL) and extracted with ether (200 mL). The organic phase was concentrated and fractionated by silica gel flash column (6-8% EtOAc in hexanes containing 0.5% HOAc) to yield the desired acid as a colorless oil (636 mg, 13%). ¹H NMR (400 MHz, CDCl₃). 7.26 (s, 1H), 6.92 (d, 1H, J=8 Hz) 6.63 (d, 1H, J=8 Hz), 4.71 (s, 2H), 2.88 (t, 2H, J=7.6 Hz), 2.63 (t, 2H, J=7.6 Hz), 0.97 (s, 9H), 0.93 (s, 9H), 0.18 (s, 6H), 0.08 (s, 6H); ¹³C NMR (100.6 MHz), 177.6, 150.4, 132.6, 132.1, 126.9, 126.7, 117.8, 60.5, 35.6, 30.1, 26.0, 25.7, 185, 18.2, −4.3, −5.4; HRMS calcd. for C₂₂H₄₀O₄Si₂ (M⁺) 424.2465. Found 424.2474.

N-Hydroxysuccimidyl 3-(3-acetyloxymethyl-4-t-butyldimethylsilyoxyphenyl)-propionate. FeCl₃6H₂O (15 mg, 0.055 mmol) was added to a solution of the acid above (200 mg, 0.48 mmol) in acetic anhydride (3.0 mL) at 0° C. and the resulting yellow solution was stirred at ambient temperature for 30 min. The mixture was diluted with ether (100 mL) and washed with H₂O (100 mL) and saturated NaHCO₃ solution (100 mL). The organic layer was collected, dried over MgSO₄ and concentrated. The resulting oil was dissolved in DMF (10 mL) and 1-ethyl-3-(3-dimethylaminopropyl)carbodiimide (EDCl) (100 mg, 0.52 mmol), N-hydroxysuccimide (65 mg, 0.57 mmol) was added. The reaction was stirred under N₂ and ambient temperature for 20 h. The reaction solution was diluted with H₂O (100 mL) and washed with ether (100 mL). The organic phase was then concentrated and fractionated by a silica gel flash column (10-45% EtOAc in hexanes) to yield the desired product as a colorless oil (51 mg, 24%). ¹H NMR (400 MHz, CDCl₃). 7.13 (s, 1H), 7.02 (d, 1H, J=8 Hz) 6.73 (d, 1H, J=8 Hz), 5.05 (s, 2H), 2.96 (t, 2H, J=7.6 Hz), 2.86 (t, 2H, J=7.6 Hz), 2.80 (s, 4H), 2.06 (s, 3H), 0.96 (s, 9H), 0.20 (s, 6H); ¹³C NMR. 170.9, 169.1, 167.9, 152.7, 131.7, 130.1, 129.2, 126.6, 118.7, 62.1, 32.8, 29.6, 25.6, 25.6, 21.0, 18.2, −4.3; HRMS calcd. for C₂₂H₃₁O₇NSi (M⁺) 449.1870. Found 449.1856.

N-Hydroxysuccimidyl diphenylacetate. A solution of EDCl (345 mg, 1.80 mmol), N-hydroxysuccimide (210 mg, 1.80 mmol) and diphenylacetic acid (318 mg, 1.50 mmol) in DMF (5.0 mL) was stirred under N₂ and ambient conditions for 20 h. The reaction solution was diluted with ether (100 mL) and washed with H₂O. The organic layer was collected and concentrated. The desired succimidyl ester was crystallized with EtOAc/hexanes as a white solid (223 mg, 48%) with mp 120-122° C. ¹H NMR (400 MHz, CDCl₃). 7.32 (m, 10H), 5.33 (s, 1H), 2.80 (s, 4H); ¹³C NMR (CDCl₃, 100.6 MHz). 168.9, 168.1, 136.7, 128.8, 128.7, 127.9, 54.0, 25.6; HRMS calcd for C₁₈H₁₆NO₄ (M+H⁺) 310.1079. Found 310.1069.

Those of ordinary skill in the art will realize that various changes may be made without departing from the spirit of the invention and therefore the invention is not limited to what is shown in the drawings and described in the specification. The disclosure of all references cited herein are hereby incorporated by reference in their entirety.

We claim:

1. A method for selective, target-activated modification of a target molecule, said method comprising:
providing a reagent comprising (i) a ligand including at least one recognition element and (ii) at least one reactive component, wherein the reagent is capable of reversible, intramolecular attachment between the ligand and the reactive component;
specifically binding the reagent with the target molecule via the at least one recognition element of the ligand; and
binding the reactive component of the reagent to the target molecule.

2. The method of claim 1, wherein a covalent bond binds the reactive component of the reagent to the target molecule.

3. The method of claim 1, wherein the intramolecular attachment between the ligand and the reactive component is via a covalent bond.

4. The method of claim 1, wherein the ligand and the target molecule each comprise nucleic acid and the specific binding is via nucleic acid hybridization.

5. The method of claim 1, wherein the at least one reactive component comprises quinone methide or imino-quinone methide.

6. The method of claim 1, wherein the ligand comprises nucleic acid.

7. The method of claim 6, wherein the nucleic acid is selected from the group consisting of DNA and RNA.

8. The method of claim 6, wherein the nucleic acid is selected from the group consisting of oligonucleotides, oligonucleotide derivates, PNA, LNA and methyl-phosphonates.

9. The method of claim 1, wherein the at least one recognition element of the ligand comprises a nucleotide.

10. The method of claim 1, wherein the target molecule comprises nucleic acid.

11. The method of claim 10, wherein the nucleic acid is selected from the group consisting of DNA and RNA.

12. The method of claim 1, wherein the ligand comprises a protein.

13. The method of claim 1, wherein the at least one recognition element of the ligand is selected from the group consisting of amino acids, amino acid analogues, peptides, analogue peptides, protein antagonists and agonists, transition state analogues and substrate analogues.

14. The method of claim 1, wherein the target molecule comprises a protein.

15. A reagent for the selective, target-activated modification of a target molecule, said reagent comprising (i) a ligand including at least one recognition element and (ii) at least one reactive component, the reagent capable of reversible, intramolecular attachment between the ligand and the reactive component;
wherein the reagent is capable of specifically binding with the target molecule via the at least one recognition element of the ligand; and
wherein the reagent is capable of binding the reactive component of the reagent to the target molecule.

16. The reagent of claim 15, wherein the reagent is capable of covalently binding the reactive component of the reagent to the target molecule.

17. The reagent of claim 15, wherein the intramolecular attachment between the ligand and the reactive component is via a covalent bond.

18. The reagent of claim 15, wherein the ligand and the target molecule each comprise nucleic acid and the specific binding is via nucleic acid hybridization.

19. The reagent of claim 15, wherein the at least one reactive component comprises quinone methide or imino-quinone methide.

20. The reagent of claim 15, wherein the ligand comprises nucleic acid.

21. The reagent of claim 20, wherein the nucleic acid is selected from the group consisting of DNA and RNA.

22. The reagent of claim 20, wherein the nucleic acid is selected from the group consisting of oligonucleotides, oligonucleotide derivates, PNA, LNA and methyl-phosphonates.

23. The reagent of claim 15, wherein the at least one recognition element of the ligand comprises a nucleotide.

24. The reagent of claim 15, wherein the target molecule comprises nucleic acid.

25. The reagent of claim 24, wherein the nucleic acid is selected from the group consisting of DNA and RNA.

26. The reagent of claim 15, wherein the ligand comprises a protein.

27. The reagent of claim 15, wherein the at least one recognition element of the ligand is selected from the group consisting of amino acids, amino acid analogues, peptides, analogue peptides, protein antagonists and agonists, transition state analogues and substrate analogues.

28. The reagent of claim 15, wherein the target molecule comprises a protein.

29. An intermediate in the selective, target-activated modification of a target molecule, said intermediate comprising the target molecule and a reagent comprising (i) a ligand including at least one recognition element and (ii) at least one reactive component, the reagent capable of reversible, intramolecular attachment between the ligand and the reactive component;
wherein the reagent is specifically bound with the target molecule via the at least one recognition element of the ligand; and
wherein the reagent is capable of binding the reactive component to the target molecule.

30. The intermediate of claim 29, wherein the reagent is capable of covalently binding the reactive component of the recognition-driven reagent to the target molecule.

31. The intermediate of claim 29, wherein the intramolecular attachment between the ligand and the reactive component is via a covalent bond.

32. The intermediate of claim 29, wherein the ligand and the target molecule each comprise nucleic acid and the specific binding is via hybridization.

33. The intermediate of claim 29, wherein the at least one reactive component comprises quinone methide or imino-quinone methide.

34. The intermediate of claim 29, wherein the ligand comprises nucleic acid.

35. The intermediate of claim 34, wherein the nucleic acid is selected from the group consisting of DNA and RNA.

36. The intermediate of claim 34, wherein the nucleic acid is selected from the group consisting of oligonucleotides, oligonucleotide derivates, PNA, LNA and methyl-phosphonates.

37. The intermediate of claim 29, wherein the at least one recognition element of the ligand comprises a nucleotide.

38. The intermediate of claim 29, wherein the target molecule comprises nucleic acid.

39. The intermediate of claim 38, wherein the nucleic acid is selected from the group consisting of DNA and RNA.

40. The intermediate of claim 29, wherein the ligand comprises a protein.

41. The intermediate of claim 29, wherein the at least one recognition element of the ligand is selected from the group consisting of amino acids, amino acid analogues, peptides, analogue peptides, protein antagonists and agonists, transition state analogues and substrate analogues.

42. The intermediate of claim 29, wherein the target molecule comprises a protein.
